(19) United States
(12) Patent Application Publication
(10) Pub. No.: US 2005/0260733 A1
(43) Pub. Date: Nov. 24, 2005

(54) Proteases and uses thereof

(75) Inventors: Edward R. LaVallie, Harvard, MA (US); Lisa A. Collins-Racie, Acton, MA (US); Christopher J. Corcoran, Arlington, MA (US); Michael J. Agostino, Andover, MA (US); Bethany A. Freeman, Belmont, MA (US); Maya Arai, Brookline, MA (US); Carl R. Flannery, Acton, MA (US); Macy X. Jin, Reading, MA (US)

Correspondence Address: NIXON PEABODY LP, 401 9TH STREET, N.W., SUITE 900, WASHINGTON, DC, 20004, US

(73) Assignee: WYETH, Cambridge, MA (US)

(21) Appl. No.: 11/106,623

(22) Filed: Apr. 15, 2005

Related U.S. Application Data
(60) Provisional application No. 60/562,687, filed on Apr. 16, 2004.

(51) Int. Cl. C12N 9/64

(52) U.S. Cl. 435/226

(57) ABSTRACT

The present invention features methods of using ADAMTS-8 proteins or their functional derivatives to cleave aggrecan or other proteoglycan molecules. The present invention also features methods for identifying ADAMTS-8 modulators that are capable of inhibiting or enhancing ADAMTS-8 proteolytic activities. In addition, the present invention features pharmaceutical compositions comprising ADAMTS-8 proteins or their derivatives or modulators. These pharmaceutical compositions can be used to treat diseases that are characterized by deficiencies or abnormalities in proteoglycan cleavage or metabolism.

This application claims the benefit and incorporates by reference the entire disclosure of U.S. Provisional Application Ser. No. 60/562,687, filed Apr. 16, 2004.

### TECHNICAL FIELD

The present invention relates to ADAMTS-8 proteins and their derivatives and modulators, and methods of using the same to treat diseases that are characterized by deficiencies or abnormalities in proteoglycan cleavage or metabolism.

### BACKGROUND

The ADAMTS (A Disintegrin And Metalloprotease with ThromboSpondin motifs) family includes at least 19 members that are related to one another on the basis of their common domain structure. In contrast to members of the ADAM family, ADAMTS proteins lack a transmembrane domain and contain at least one thrombospondin 1-like motif. A typical ADAMTS protein contains, from N- to C-terminus, a signal sequence, a prodomain, a metalloprotease catalytic domain, a disintegrin-like domain, a central thrombospondin type I repeat, a cysteine-rich domain, and a spacer domain. See Cal, et al., GENE, 283:49-62 (2002). Many ADAMTS proteins also include one or more thrombospondin 1-like repeats following the spacer domain. ADAMTS proteins are capable of associating with components of the extracellular matrix through interactions within the spacer domain and the thrombospondin 1-like repeat(s). See Kuno and Matsushima, J. BIOL. CHEM., 273:13912-13917 (1998).

The physiological roles of a small subset of ADAMTS family members have been elucidated, and in some cases aberrant expression has been implicated in human disease. ADAMTS-2, ADAMTS-3, and ADAMTS-14 reportedly function as procollagenases. ADAMTS-2 has been identified as a procollagen I N-proteinase (pNPI) responsible for processing of type I and type II procollagens. The absence of type I procollagen processing results in the accumulation of collagen fibrils that retain the amino-terminal propeptide (pN-collagen I). Fibrils constructed from pN-collagen I do not provide normal levels of tensile strength, thereby causing disease-associated connective tissue defects. Ehlers-Danlos syndrome type VIIC is a human recessive genetic disorder caused by the inability to process type 1 procollagen to collagen, resulting in loss of joint integrity and fragility of the skin. A related disease seen in cattle, sheep, and some breeds of cat is called dermatosparaxis (“tearing of skin”). Both of these diseases have been linked to loss of ADAMTS-2 activity. Residual amino-propeptide cleavage of type 1 collagen in the absence of ADAMTS-2 activity led to the discovery that ADAMTS-14 is also capable of cleaving type I collagen in vitro. ADAMTS-3 has been proposed to be the major procollagen II N-propeptidase. ADAMTS-13 has been identified as a plasma protease that cleaves von Willebrand factor (vWF) at a specific Tyr-Met bond within the A2 domain. Thrombotic thrombocytopenic purpura (TTP) is a syndrome characterized by microvascular thrombosis, low platelet count, and anemia. It is postulated that lack of appropriate cleavage of large vWF (UL-vWF) multimers released from endothelial cells may result in TTP. Genetic analysis of 4 familial TTP pedigrees demonstrated that mutations in the ADAMTS-13 gene were largely responsible for this disorder.

ADAMTS-1, ADAMTS-4, ADAMTS-5, and ADAMTS-9 have been shown to be capable of cleaving the extracellular matrix proteoglycans with varying degrees of efficiency. For instance, ADAMTS-1, ADAMTS-4, and ADAMTS-5 can cleave the Glu³⁷³-Ala³⁷⁴ bond in the interglobular domain (IGD) of aggrecan. See Caterson, et al., MATRIX BIOLOGY, 19:333-344 (2000). This proteolytic activity is referred to as aggrecanase activity, and the Glu³⁷³-Ala³⁷⁴ bond is known as the aggrecanase cleavage site. A protein possessing the aggrecanase activity is called an aggrecanase. The Glu³⁷³-Ala³⁷⁴ bond is hydrolyzed in vivo during degenerative joint diseases such as osteoarthritis. Evidence suggests that aggrecanases are responsible for primary cleavage of the IGD during cartilage degradation. See Caterson, et al., supra. ADAMTS4 was also found to play a role in the cleavage of brevican, a proteoglycan abundant in adult brain, and, together with ADAMTS1, has been shown to cleave versican.

ADAMTS-8, also known as Meth2, has been implicated in angiogenesis. Studies have shown that recombinant ADAMTS-8 can inhibit endothelial cell proliferation in vitro, and vascularization in in vivo assays. See, for example, Vázquez, et al., J. BIOL. CHEM., 274:23349-23357 (1999). ADAMTS-8 appears to disrupt angiogenesis in vitro and in vivo more efficiently than thrombospondin-1 or endostain, but less efficiently than ADAMTS-1. No proteolytic activity has been identified for ADAMTS-8.

### SUMMARY OF THE INVENTION

The present invention features the use of isolated ADAMTS-8 proteins to cleave proteoglycans. Methods suitable for this purpose comprise contacting a proteoglycan molecule with an isolated ADAMTS-8 protein which cleaves the proteoglycan molecule. In many embodiments, the proteoglycan molecule being cleaved is an aggrecan molecule, and the isolated ADAMTS-8 protein cleaves the aggrecan molecule at the Glu³⁷³-Ala³⁷⁴ bond. The ADAMTS-8 proteins employed in the present invention can be full-length, mature ADAMTS-8 proteins. In one example, the ADAMTS-8 protein employed comprises or consists of amino acids 214-890 of SEQ ID NO:28. In another example, the ADAMTS-8 protein employed is encoded by GenBank Accession No. AF060153 but lacks signal peptide and prodomain.

The present invention also features the use of isolated ADAMTS-8 derivatives to cleave proteoglycans. These ADAMTS-8 derivatives comprise an ADAMTS-8 metalloprotease catalytic domain and possess the proteoglycan cleavage activities (e.g., aggrecanase activity) of the full-length, mature ADAMTS-8 proteins. Contacting such an ADAMTS-8 derivative with a proteoglycan molecule (e.g., an aggrecan molecule) cleaves the proteoglycan molecule. In one example, the ADAMTS-8 metalloprotease catalytic domain employed in the present invention comprises or consists of amino acids 214-439 of SEQ ID NO:28. An ADAMTS-8 derivative can further include an ADAMTS-8 disintegrin-like domain and/or an ADAMTS-8 central thrombospondin type I repeat.

ADAMTS-8 derivatives suitable for the present invention can be prepared by any conventional means. In many cases, the ADAMTS-8 derivatives do not include signal peptide or prodomain. The ADAMTS-8 derivatives can be prepared from full-length ADAMTS-8 proteins through deletion, insertion or substitution of selected amino acid residues. In one embodiment, an ADAMTS-8 derivative employed in the present invention comprises or consists of amino acids 214-588 of SEQ ID NO:28. ADAMTS-7 or ADAMTS-9 derivatives consisting of the corresponding amino acid sequences have been shown to retain the aggrecanase activity of the original full-length proteins.

In another aspect, the present invention features the use of recombinantly-produced ADAMTS-8 proteins or their derivatives to cleave proteoglycans. Methods suitable for this purpose comprise expressing an ADAMTS-8 protein or a derivative thereof from a recombinant expression vector. The expressed ADAMTS-8 protein or derivative cleaves a proteoglycan molecule (e.g., an aggrecan molecule) upon contact. Any ADAMTS-8 protein or derivative described herein can be recombinantly produced. In many embodiments, recombinant vectors encoding ADAMTS-8 proteins or derivatives are expressed in mammalian cells which secrete the expressed proteins or derivatives into culture media or extracellular matrix regions. In one example, a recombinant expression vector employed in the present invention comprises a sequence encoding amino acids 214-890 of SEQ ID NO:28. In another example, a recombinant expression vector employed in the present invention comprises a sequence encoding amino acids 214-588 of SEQ ID NO:28. In still another example, a recombinant expression vector employed in the present invention comprises the protein coding sequence of GenBank Accession No. AF060153.

The proteoglycans being cleaved according to the present invention can be located in a tissue, a tissue culture, or a cell culture. An isolated or recombinantly-produced ADAMTS-8 protein or derivative can be delivered to a tissue site by any conventional means, such as by parenteral, intravenous, topical, intradermal, transdermal or subcutaneous administration, or by introducing an expression vectors encoding an ADAMTS-8 protein or derivative into selected cells at the tissue site.

The present invention further features methods for the identification of ADAMTS-8 modulators. These methods comprise:
contacting an ADAMTS-8 protein or derivative with a proteoglycan molecule (e.g., an aggrecan molecule) in the presence or absence of an agent of interest; and
measuring the proteoglycan cleavage activity (e.g., aggrecanase activity) of the ADAMTS-8 protein or derivative in the presence or absence of the agent.
   A change in the proteoglycan cleavage activity (e.g., aggrecanase activity) in the presence of the agent, as compared to in the absence of said agent, indicates that the agent is capable of modulating the proteoglycan cleavage activity of the ADAMTS-8 protein or derivative. Any ADAMTS-8 protein or derivative described herein can be used for screening for ADAMTS-8 modulators. The modulators identified according to the present invention can inhibit (e.g., reduce or eliminate) or enhance the proteoglycan cleavage activity (e.g., aggrecanase activity) of an ADAMTS-8 protein.

The present invention also features the use of ADAMTS-8 modulators to treat diseases that are characterized by deficiencies or abnormalities in proteoglycan cleavage (e.g., aggrecan cleavage). Methods suitable for this purpose comprise administering a therapeutically effective amount of an ADAMTS-8 modulator to a mammal in need thereof. Any route of administration can be used, provided that the ADAMTS-8 modulator can reach the desired tissue site(s) and is effective in altering proteoglycan cleavage activities at the site(s). Any ADAMTS-8 modulator identified by the present invention can be used for treating proteoglycan deficiencies or abnormalities.

The proteoglycan cleavage activities at a tissue site can also be modulated by introducing an isolated ADAMTS-8 protein or derivative, or by expressing a recombinant ADAMTS-8 protein or derivative at the site. Moreover, proteoglycan cleavage activities in an extracellular matrix region can be modulated by inhibiting the expression of ADAMTS-8 in selected cells in the region. Methods suitable for this purpose include, but are not limited to, introducing or expressing an ADAMTS-8 RNAi or antisense sequence in the selected cells. In many cases, the RNAi or antisense sequence employed is specific for the ADAMTS-8 gene and incapable of inhibiting the expression of other protease genes.

The present invention also features pharmaceutical compositions comprising ADAMTS-8 proteins or their derivatives or modulators.

Other features, objects, and advantages of the present invention are apparent in the detailed description that follows. It should be understood, however, that the detailed description, while indicating preferred embodiments of the present invention, is given by way of illustration only, not limitation. Various changes and modifications within the scope of the invention will become apparent to those skilled in the art from the detailed description.

### BRIEF DESCRIPTION OF THE DRAWINGS

The drawings are provided for illustration, not limitation.

FIG. 1 illustrates a phylogenetic tree of ADAMTS family members. Amino acid sequences of multiple ADAMTS proteins were compared using CLUSTALW, and displayed using TreeView. The phylogram groups the proteins together based upon sequence relatedness.

FIG. 2A shows a 10% SDS-PAGE of protein fractions from Strep-tag® purification (IBA, Germany) of ADAMTS-8 proteins isolated from CHO conditioned media. The SDS-PAGE was stained with Coomassie Brilliant Blue. Lanes: 1, CHO cell conditioned medium; lane 2, flow-through fraction (filtrate) from ultrafiltration; lane 3, concentrated ultrafiltration retentate fraction; lane 4, Streptactin column flow-through fraction; lanes 5-9, Streptactin column wash fractions; lanes 10-15, Streptactin column elution fractions.

FIG. 2B is a Western blot of the SDS-PAGE of FIG. 2A using an anti Strep-Tag II polyclonal antiserum (IBA).

FIG. 3A depicts a multiple tissue expression array of mRNA from 76 different human tissues, probed with a cDNA fragment probe from human ADAMTS-8 gene.

FIG. 3B indicates the sources of mRNA used by the multiple tissue expression array of FIG. 3A. Blank boxes indicate that no mRNA was spotted at those coordinates. Tissues with high relative abundance of ADAMTS-8 mRNA are lung (A8), aorta (B4), and fetal heart (B11), with lower levels of ADAMTS-8 mRNA detectable in appendix (G5) and various regions of the brain (A1-G1, C3-H3, and B3).

FIG. 4 demonstrates a histogram of ADAMTS-8 mRNA expression levels in human clinical samples of disease-free and osteoarthritic (OA) cartilage determined by real-time PCR. Samples W-04 through W-13 represent non-OA affected (“Disease-Free”) knee articular cartilage. Samples 77M-96M represent visually unaffected regions of late-stage OA articular cartilage (“Mild OA”). Samples 88S-98S represent severely affected regions of late-stage OA articular cartilage (“Severe OA”). ADAMTS-8 mRNA abundance in each sample was reported as a normalized value, by dividing the averaged data determined for ADAMTS-8 by the averaged data determined for GAPDH in the same sample.

FIG. 5 shows the results of competitive inhibition ELISAs using monoclonal antibody AGG-C1. Streptavadin-coated microtiter plates were coated with biotinylated aggc1 peptide. Inhibition analyses were performed using the following competitors: synthetic peptide GGLPLPRNITEGE (SEQ ID NO:22, closed squares), GGLPLPRNITEGEARGSVILTVK-CONH₂ (SEQ ID NO:23, open squares), ADAMTS-4 digested aggrecan (closed circles), and undigested aggrecan (open circles).

FIG. 6A is a Western blot of ADAMTS-4 and ADAMTS-8 digested bovine aggrecan using monoclonal antibody BC-3. Bovine aggrecan was incubated without or with ADAMTS-4 or ADAMTS-8 for 16 h at 37° C. Digestion products were separated by SDS-PAGE and visualized by Western immunoblotting using monoclonal antibody BC-3. Lane 1, no enzyme added; lane 2, ADAMTS-4 digested aggrecan (1:20 molar ratio enzyme:substrate); lanes 3-7, ADAMTS-8 digested aggrecan at molar ratio enzyme:substrate shown above each lane. The migration positions of globular protein standards are shown to the left of the blot.

FIG. 6B is a Western blot of ADAMTS-8 digested bovine aggrecan using monoclonal antibody AGG-C1. Bovine aggrecan was incubated with either no enzyme, or with increasing molar ratios of ADAMTS-8 for 16 h at 37° C. Digestion products were separated SDS-PAGE and visualized by Western immunoblotting using monoclonal antibody AGG-C1. The relative molar ratio of enzyme:substrate in each digest is indicated.

FIG. 6C depicts a Western blot of ADAMTS-4 digested bovine aggrecan using monoclonal antibody AGG-C1. Bovine aggrecan (12.5 pmol) was incubated with either no enzyme, or with 0.05 ng, 0.1 ng, 0.25 ng, 0.5 ng, or 1 ng of ADAMTS-4, respectively, for 16 h at 37° C. Digestion products were separated in SDS-PAGE and visualized by Western immunoblotting using AGG-C1. The relative molar ratio of enzyme:substrate in each digest is indicated.

FIG. 7 shows the result of competitive inhibition ELISA for aggrecanase activity. The standard curve was generated by incubating bovine aggrecan with increasing amounts of recombinant ADAMTS-4 for 16 h at 37° C. followed by addition of monoclonal antibody AGG-C1 to each digest. It requires approximately 1 ng of ADAMTS-4 to generate an amount of aggrecan cleavage product that results in 45% inhibition in the competitive inhibition ELISA.

### DETAILED DESCRIPTION

The present invention features the use of ADAMTS-8 proteins or their derivatives to cleave proteoglycan molecules. The present invention also features methods for identifying ADAMTS-8 modulators that are capable of inhibiting or enhancing ADAMTS-8 proteolytic activities. In addition, the present invention provides pharmaceutical compositions comprising ADAMTS-8 proteins or their derivatives or modulators. These pharmaceutical compositions can be used to treat conditions that are characterized by deficiencies or abnormalities in proteoglycan cleavage or metabolism.

Various aspects of the invention are described in detail in the following sections. The use of sections is not meant to limit the invention. Each section can apply to any aspect of the invention. In this application, the use of “or” means “and/or” unless stated otherwise.

I. ADAMTS-8 Proteins and Their Functional Derivatives

The present invention features the use of mature ADAMTS-8 proteins for the cleavage of aggrecan or other proteoglycan molecules. Mature ADAMTS-8 proteins lack signal peptide and prodomain. Examples of suitable mature ADAMTS-8 proteins include, but are not limited to, full-length mature ADAMTS-8 proteins (e.g., the furin-processed ADAMTS-8 protein encoded by GenBank Accession No. AF060153), and mature ADAMTS-8 isoforms produced by alternative RNA splicing or proteolytic processing of the ancillary domains. Alternative RNA splicing, which results in deletion of one or more C-terminal thrombospondin 1-like repeats, has been observed for certain members of the ADAMTS family. Proteolytic removal of C-terminal ancillary domains during the maturation process has also been reported for certain ADAMTS family members.

The present invention also contemplates the use of unprocessed ADAMTS protein for the cleavage of aggrecan or other proteoglycan molecules. These unprocessed proteins include signal peptide or prodomain. In many cases, the unprocessed ADAMTS-8 proteins are recombinantly expressed in suitable host cells and secreted into culture media or extracellular matrix regions. These secreted proteins typically lack the signal sequence. These proteins can be further proteolytically processed to remove the prodomain.

The ADAMTS-8 proteins employed in the present invention can be naturally-occurring proteins, such as that encoded by GenBank Accession No. AF060153 or its naturally-occurring proteolytic products. In one example, the ADAMTS-8 protein employed in the present invention comprises amino acids 214-890 of SEQ ID NO:28.

The present invention also features the use of variants of naturally-occurring ADAMTS-8 proteins for the cleavage of aggrecan or other proteoglycan molecules. These variants retain the proteoglycan cleavage activities (e.g., aggrecanase activity) of the original proteins. The amino acid sequence of a variant is substantially identical to that of the original protein. In one example, the amino acid sequence of a variant has at least 60%, 65%, 70%, 75%, 80%, 85%, 90%, 95%, 99%, or more global sequence identity or similarity to the original protein. Sequence identity or similarity can be determined using various methods known in the art. For instance, sequence identity or similarity can be determined using standard alignment algorithms, such as Basic Local Alignment Tool (BLAST) described in Altschul, et al., J. MOL. BIOL., 215:403-410 (1990), the algorithm of Needleman, et al., J. MOL. BIOL., 48:444-453 (1970), the algorithm of Meyers, et al., COMPUT. APPL. BIOSCI., 4:11-17(1988), and dot matrix analysis. Softwares suitable for this purpose include, but are not limited to, BLAST programs provided by the National Center for Biotechnology Information (Bethesda, Md.) and MegAlign provided by DNASTAR, Inc. (Madison, Wis.). In one instance, the sequence identity or similarity is determined using the Genetics Computer Group (GCG) programs GAP (Needleman-Wunsch algorithm). Default values assigned by the programs can be employed (e.g., the penalty for opening a gap in one of the sequences is 11 and for extending the gap is 8). Similar amino acids can be defined using the BLOSUM62 substitution matrix.

ADAMTS-8 protein variants can be naturally-occurring, such as by allelic variations or polymorphisms, or deliberately engineered. In many examples, conservative amino acid substitutions can be introduced into a protein sequence without significantly changing the structure or biological activity of the protein. Conservative amino acid substitutions can be made on the basis of similarity in polarity, charge, solubility, hydrophobicity, hydrophilicity, or the amphipathic nature of the residues. For instance, conservative amino acid substitutions can be made among amino acids with basic side chains, such as lysine (Lys or K), arginine (Am or R) and histidine (His or H); amino acids with acidic side chains, such as aspartic acid (Asp or D) and glutamic acid (Glu or E); amino acids with uncharged polar side chains, such as asparagine (Asn or N), glutamine (Gln or Q), serine (Ser or S), threonine (Thr or T), and tyrosine (Tyr or Y); and amino acids with nonpolar side chains, such as alanine (Ala or A), glycine (Gly or G), valine (Val or V), leucine (Leu or L), isoleucine (Ile or I), proline (Pro or P), phenylalanine (Phe or F), methionine (Met or M), tryptophan (Trp or W) and cysteine (Cys or C). Other suitable amino acid substitutions are illustrated in Table 1.

TABLE 1
Exemplary Amino Acid Substitutions

| | | More |
|---|---|---|
| Original | | Conservative |
| Residues | Exemplary Substitutions | Substitutions |
| Ala (A) | Val, Leu, Ile | Val |
| Arg (R) | Lys, Gln, Asn | Lys |
| Asn (N) | Gln | Gln |
| Asp (D) | Glu | Glu |
| Cys (C) | Ser, Ala | Ser |
| Gln (Q) | Asn | Asn |
| Gly (G) | Pro, Ala | Ala |
| His (H) | Asn, Gln, Lys, Arg | Arg |
| Ile (I) | Leu, Val, Met, Ala, Phe, Norleucine | Leu |
| Leu (L) | Norleucine, Ile, Val, Met, Ala, Phe | Ile |
| Lys (K) | Arg, 1,4 Diamino-butyric Acid, Gln, Asn | Arg |
| Met (M) | Leu, Phe, Ile | Leu |
| Phe (F) | Leu, Val, Ile, Ala, Tyr | Leu |
| Pro (P) | Ala | Gly |
| Ser (S) | Thr, Ala, Cys | Thr |
| Thr (T) | Ser | Ser |
| Trp (W) | Tyr, Phe | Tyr |
| Tyr (Y) | Trp, Phe, Thr, Ser | Phe |
| Val (V) | Ile, Met, Leu, Phe, Ala, Norleucine | Leu |

Non-naturally-occurring amino acid residues can also be used for substitutions. These amino acid residues are typically incorporated by chemical peptide synthesis rather than by synthesis in biological systems.

In addition, ADAMTS-8 variants can include amino acid substitutions to increase the stability of the molecules. Other desirable amino acid substitutions (whether conservative or non-conservative) can also be introduced into ADAMTS-8 proteins. For instance, amino acid residues important to a proteolytic activity of an ADAMTS-8 protein can be identified. Substitutions capable of increasing or decreasing that proteolytic activity can be selected.

Moreover, ADAMTS-8 variants can include modifications of glycosylation sites. These modifications can involve O-linked or N-linked glycosylation sites. For instance, the amino acid residues at asparagine-linked glycosylation recognition sites can be substituted or deleted, resulting in partial glycosylation or complete abolishment of glycosylation. The asparagine-linked glycosylation recognition sites typically comprise tripeptide sequences that are recognized by appropriate cellular glycosylation enzymes. These tripeptide sequences can be, for example, asparagine-X-threonine or asparagine-X-serine, where X is usually any amino acid. A variety of amino add substitutions or deletions at one or both of the first or third amino acid positions of a glycosylation recognition site (or amino acid deletion at the second position) can result in non-glycosylation at the modified tripeptide sequence. Additionally, bacterial expression also results in production of non-glycosylated proteins, even if the glycosylation sites are left unmodified.

Other types of modifications can also be introduced into an ADAMTS-8 variant. These modifications can be introduced by naturally-occurring processes, such as posttranslational modifications, or by artificial or synthetic processes. Modifications may occur anywhere in the polypeptide, including the backbone, the amino acid side chains, and the amino or carboxyl termini. The same type of modification can be present in the same or varying degrees at several sites in a variant. A variant can also include many different types of modifications. Modifications suitable for this invention include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, ubiquitination, or any combination thereof. A polypeptide variant can be branched (e.g., as a result of ubiquitination), or cyclic, with or without branching.

An ADAMTS-8 variant employed in the present invention can be substantially identical to the original ADAMTS-8 protein in one or more regions, but divergent in other regions. An ADAMTS-8 variant can retain the overall domain structure of the original ADAMTS-8 protein. In one embodiment, a variant is prepared by modifying at least 1, 2, 3, 4, 5, 10, 15, 20, 25, 30, 35, 40, 45, 50, or more amino acid residues of a naturally-occurring ADAMTS-8 sequence. Exemplary modifications include, but are not limited to, substitutes, deletions, and insertions. The substitutions can be conservative, non-conservative, or both. These modifications do not significantly affect the proteolytic activities (e.g., aggrecanase activity) of the original protein. For instance, a variant can retain at least 10%, 20%, 30%, 40%, 50%, 60%, 70%, 80%, 90%, or more of a proteolytic activity (e.g., aggrecanase activity) of the original ADAMTS-8 protein. A variant can also have an improved proteolytic activity (e.g., improved aggrecanase activity) as compared to the original ADAMTS-8 protein.

The present invention further features the use of ADAMTS-8 derivatives for the cleavage of aggrecan or proteoglycan molecules. These ADAMTS-8 derivatives are modified ADAMTS-8 proteins with deletions or modification of one or more amino acid residues. In one example, an ADAMTS-8 derivative includes deletion of a substantial portion of an ancillary domain of a full-length ADAMTS-8 protein. In another example, an ADAMTS-8 derivative includes deletion of the spacer domain and the C-terminal thrombospondin 1-like repeat from a full-length ADAMTS-8 protein. Any region after the spacer domain and the C-terminal thrombospondin 1-like repeat can also be deleted.

In one embodiment, an ADAMTS-8 derivative employed in the present invention includes deletion of a substantial portion of the amino acid residues located after Phe⁵⁸⁸ of SEQ ID NO:28. ADAMTS-7 or ADAMTS-9 truncations with deletion of the corresponding sequences have been shown to retain the aggrecanase activity of the original proteins. The amino acid residues deleted from a full-length ADAMTS-8 protein can include, without limitation, at least 30%, 40%, 50%, 60%, 70%, 80%, 90%, 95%, 96%, 97%, 98%, 99%, or 100% of the amino acid residues that are located C-terminal to Phe⁵⁸⁸. The deleted amino acid residues can be selected from the cysteine-rich domain, the spacer domain, the C-terminal thrombospondin 1-like repeat, or any region located therebetween or thereafter. The deleted residues can be contiguous or noncontiguous. In one example, an ADAMTS-8 derivative comprises or consists of amino acids 214-588 of SEQ ID NO:28.

Amino acid residues in the N-terminal region of an ADAMTS-8 protein can also be modified. For instance, certain selected residues in the signal sequence, the prodomain, the metalloprotease catalytic domain, the disintegrin-like domain, or the central thrombospondin type I repeat can be deleted or otherwise modified without significantly reducing the proteolytic activities (e.g., aggrecanase activity) of the ADAMTS-8 protein.

Additional polypeptides can be fused to the N- or C-terminus of an ADAMTS-8 protein or its functional derivatives. Non-limiting examples of these polypeptides include peptide tags, enzymes, antibodies, receptors, ligand/receptor binding proteins, or combinations thereof. Antibodies suitable for this purpose include, but are not limited to, polyclonal, monoclonal, mono-specific, poly-specific, non-specific, humanized, human, single-chain, chimeric, synthetic, recombinant, hybrid, mutated, grafted, or in vitro generated antibodies. Antibody fragments can also be used. Examples of these antibody fragments include, but are not limited to, Fab, F(ab′)₂, Fv, Fd, or dAb.

Peptide tags can also be added to an ADAMTS-8 protein or its derivatives. Suitable peptide tags include, but are not limited to, the Strep-tag® (IBA), the poly-histidine or poly-histidine-glycine tag, the FLAG epitope tag, the KT3 epitope peptide, the flu HA tag polypeptide, the c-myc tag, the Herpes simplex glycoprotein D, beta-galactosidase, maltose binding protein, streptavidin tag, tubulin epitope peptide, the T7 gene 10 protein peptide tag, and glutathione S-transferase. Antibodies against these peptide tags can be readily obtained from a variety of commercial sources. Representative antibodies include antibody 12CA5 against the flu HA tag polypeptide, and the 8F9, 3C7, 6E10, G4, B7 and 9E10 antibodies against the c-myc tag. Peptide linkers can be added between a peptide tag and the original protein to enhance the accessibility of the peptide tag.

Proteolytically cleavable site(s) can be introduced between an added polypeptide and the original protein. These cleavable sites allow separation of the original protein from the added polypeptide. Enzymes suitable for this purpose include, but are not limited to, Factor Xa, thrombin, and enterokinase.

The added polypeptides can be used to facilitate protein purification, detection, immobilization, folding or targeting, or serve other desired purposes. These polypeptides can also be used to increase the expression, solubility, or stability of the fusion proteins. In many embodiments, the added polypeptides do not significantly affect the proteolytic activities (e.g., aggrecanase activity) of the fusion proteins.

II. Polynucleotides Encoding ADAMTS-8 Proteins or Their Functional Derivatives

Polynucleotides encoding ADAMTS-8 proteins or their derivatives can be prepared using a variety of methods. These polynucleotides can be DNA, RNA, or other expressible nucleic acid molecules. They can be single-stranded or double-stranded.

In one embodiment, GenBank Accession No. AF060153 is used for the preparation of coding sequences of ADAMTS-8 proteins or their derivatives. Deletions or other modifications can be introduced into the protein coding sequence of GenBank Accession No. AF060153 using standard recombinant DNA techniques. Exemplary DNA deletion/modification techniques include, but are not limited to, PCR-mediated mutagenesis, oligonucleotide-directed “loop-out” mutagenesis, PCR overlap extension, time-controlled digestion with exonuclease III, the megaprimer procedure, inverse PCR, and automated DNA synthesis.

Deletion libraries can also be used. These deletion libraries include coding sequences for N-terminal, C-terminal, or internal deleted ADAMTS-8 proteins. Exemplary methods for the construction of deletion libraries include, but are not limited to, that described in Pues, et al., NUCLEIC ACIDS RES., 25:1303-1305 (1997). Commercial deletion kits, such as the EZ::TN Plasmid-Based Deletion Machine and the pWEB::TNC™ Deletion Cosmid Transposition Kit (Epicentre, Madison, Wis.), can also be used to generate ADAMTS-8 deletion libraries. Deletions that retain the proteolytic activity of the original ADAMTS-8 protein can be selected.

The polynucleotides employed in the present invention can be modified to increase their stabilities in vivo. Possible modifications include, but are not limited to, the addition of flanking sequences at the 5′ or 3′ end; the use of phosphorothioate or 2-o-methyl instead of phosphodiesterase linkages in the backbone; and the inclusion of nontraditional bases such as inosine, queosine and wybutosine, as well as acetyl-, methyl-, thio-, or other modified forms of adenine, cytidine, guanine, thymine and uridine.

The present invention also features expression vectors that encode ADAMTS-8 proteins or their functional derivatives. These expression vectors comprise 5′ or 3′ untranslated regulatory sequences operably linked to a protein coding sequence that encodes an ADAMTS-8 protein or a functional derivative thereof. The design of expression vectors depends on such factors as the choice of the host cells and the desired expression levels. Non-limiting examples of suitable expression vectors include bacterial expression vectors, yeast expression vectors, insect cell expression vectors, and mammalian expression vectors. Viral vectors can also be used, such as retroviral, lentiviral, adenoviral, adeno-associated viral, herpes viral, alphavirus, astrovirus, coronavirus, orthomyxovirus, papovavirus, paramyxovirus, parvovirus, picornavirus, poxvirus, or togavirus vectors. An expression vector employed by the present invention can be controlled by either a constitutive or an inducible promoter.

The present invention also contemplates the use of tissue-specific or developmentally-regulated promoters. Examples of suitable tissue-specific promoters include, but are not limited to, cartilage-specific promoters, brain-specific promoters, lung-specific promoters, aorta-specific promoters, appendix-specific promoters, liver-specific promoters, lymphoid-specific promoters, pancreas-specific promoters, mammary gland-specific promoters, chondrocyte-specific promoters, neuron-specific promoters, glial cell-specific promoters, and T cell-specific promoters, Examples of developmentally-regulated promoters include, but, are not limited to, the α-fetoprotein promoter. The use of tissue-specific or developmentally-regulated promoters allows selected expression of ADAMTS-8 proteins or their derivatives in predetermined tissues or at specific developmental stages.

Regulatable expression systems can also be used for the expression of ADAMTS-8 proteins or their derivatives. Systems suitable for this purpose include, but are not limited to, the Tet-on/off system, the Ecdysone system, the Progesterone system, and the Rapamycin system.

III. Expression and Purification of ADAMTS-8 Proteins or Their Functional Derivatives

Expression vectors encoding ADAMTS-8 proteins or their functional derivatives can be stably or transiently introduced into host cells for expression. The expressed proteins can be isolated from the host cells using conventional means. Host cells suitable for this purpose include, but are not limited to, eukaryotic cells (e.g., mammalian cells, insect cells, or yeast) and prokaryotic cells (e.g., bacteria). Non-limiting examples of suitable eukaryotic host cells include Chinese hamster ovary cells (CHO), HeLa cells, COS cells, 293 cells, and CV-1 cells. Eukaryotic host cells usually provide desired post-translational modifications, such as glycosylation, for the expressed proteins. Non-limiting examples of suitable prokaryotic host cells include E. coli (e.g., HB101, MC1061), B. subtilis, and Pseudomonas. The host cells employed in the present invention can be cell lines, primary cell cultures, or tissue cultures. They can also be cells in transgenic or chimeric animals. The selection of suitable host cells and methods for culture, transfection/transformation, amplification, screening, and product production and purification is a matter of routine design within the level of ordinary skill in the art.

In one embodiment, an ADAMTS-8 protein or a functional derivative thereof is expressed in mammalian host cells which secrete the expressed protein into the culture medium. The secreted product can be isolated or purified using standard isolation/purification techniques, such as affinity chromatography (including immunoaffinity chromatography), ionic exchange chromatography, hydrophobic interaction chromatography, size-exclusion chromatography, HPLC, protein precipitation (including immunoprecipitation), differential solubilization, electrophoresis, centrifugation, crystallization, or any combination thereof. Purification tags, such as streptavidin tag, FLAG tag, poly-histidine tag, or glutathione S-transferase, can be used to facilitate the isolation of the expressed protein. Purification tags may be cleaved from the expressed protein after its purification. Purification tags can also be used for the isolation or purification of non-secretory ADAMTS-8 proteins from cell lysates.

In anther embodiment, an ADAMTS-8 protein or a functional derivative thereof is expressed in prokaryotic host cells and concentrated in the inclusion bodies of these cells. The concentrated protein can be solubilized from the inclusion bodies, refolded, and then isolated using the methods described above.

An isolated ADAMTS-8 protein or its derivative can be analyzed or verified using standard techniques such as SDS-PAGE or immunoblots. The isolated protein can also be analyzed by protein sequencing or mass spectroscopy. In one example, a protein band of interest in an SDS-PAGE is excised manually from the gel, and then reduced, alkylated and digested with trypsin or endopeptidase Lys-C (Promega, Madison, Wis.). The digestion can be conducted in situ using an automated in-gel digestion robot. After digestion, the peptide extracts can be concentrated and separated by microelectrospray reversed phase HPLC. Peptide analyses can be done on a Finnigan LCQ ion trap mass spectrometer (ThermoQuest, San Jose, Calif.). Automated analysis of MS/MS data can be performed using the SEQUEST computer algorithm incorporated into the Finnigan Bioworks data analysis package (ThermoQuest, San Jose, Calif.).

The present invention also features the expression of ADAMTS-8 proteins or their derivatives in cell-free transcription and translation systems. Suitable cell-free expression systems include, but are not limited to, wheat germ extracts, reticulocyte lysates, and HeLa nuclear extracts. The expressed proteins can be isolated or purified using the methods described above.

IV. Detection of Proteolytic Activities

Aggrecanase activity can be evaluated using the fluorescent peptide assay, the neoepitope Western blot, the aggrecan ELISA, or the activity assay. The first two assays are suitable for detecting the cleavage capability at the Glu³⁷³-Ala³⁷⁴ bond in the IGD of aggrecan.

In the fluorescent peptide assay, an ADAMTS-8 protein (or a derivative thereof) is incubated with a synthetic peptide which contains the amino acid sequence at the aggrecanase cleavage site. Either the N-terminus or the C-terminus of the synthetic peptide is labeled with a fluorophore and the other terminus includes a quencher. Cleavage of the peptide separates the fluorophore and quencher, thereby eliciting fluorescence. Relative fluorescence can be used to determine the relative aggrecanase activity of the protein.

In the neoepitope Western blot, an ADAMTS-8 protein (or a derivative thereof) is incubated with intact aggrecan. The cleavage products are then subject to several biochemical treatments before being separated by an SDS-PAGE. The biochemical treatments include, for example, dialysis, chondroitinase treatment, lyophilization, and reconstitution. Protein samples in the SDS-PAGE are transferred to a membrane (such as a nitrocellulose paper), and stained with a neoepitope specific antibody. The neoepitope antibody specifically recognizes a new N- or C-terminal amino acid sequence exposed by proteolytic cleavage of aggrecan. The antibody does not bind to such an epitope on the original or uncleaved molecule. Suitable neoepitope antibodies include, but are not limited to, MAb BC-13, MAb BC-3, and the I19C antibody. See, e.g., Caterson, et al., supra; and Hashimoto, et al., FEBS LETTERS, 494:192-195 (2001). In one example, cleaved aggrecan fragments are visualized using an alkaline phosphatases-conjugated secondary antibody and nitroblue tetrazolium chromogen and bromochloroindolyl phosphate substrate (NBT/BCIP). Relative density of the bands is indicative of relative aggrecanase activity.

The aggrecan ELISA can be used to detect any cleavage in an aggrecan molecule. In this assay, an ADAMTS-8 protein (or a derivative thereof) is incubated with intact aggrecan which has been previously adhered to plastic wells. The wells are washed and then incubated with an antibody that detects aggrecan. The wells are developed with a secondary antibody. If the original amount of aggrecan remains in the wells, the antibody staining would be dense. If aggrecan is digested by the ADAMTS-8 protein (or its derivative), the attached aggrecan molecule will come off the wells, thereby reducing the subsequent staining by the antibody. This assay can detect whether an ADAMTS-8 protein (or a derivative thereof) is capable of cleaving aggrecan. The relative cleavage activity can also be determined using this assay.

In the activity assay, microtiter plates are first coated with hyaluronic acid (ICN), followed by chondroitinase-treated bovine aggrecan. Chondroitinase can be obtained, for example from Seikagaku Chemicals. The culture medium containing an ADAMTS-8 protein (or a derivative thereof) is added to the aggrecan-coated plates. Aggrecan cleaved at the Glu³⁷³-Ala³⁷⁴ within the IGD is washed away. The remaining uncleaved aggrecan can be detected with the 3B3 antibody (ICN), followed by anti-IgM-HRP secondary antibody (Southern Biotechnology). Final color development can be obtained using, for example, 3,3″,5,5″ tetramethylbenzidine (TMB, BioFx Laboratories).

Proteolytic activities against brevican, versican, neurocan, or other proteoglycans or extracellular matrix proteins can also be evaluated using conventional means. See, for example, Somerville, et al., J. BIOL. CHEM., 278:9503-9513 (2003) (describing assays for evaluating versicanase activities). These methods typically involve contacting an ADAMTS-8 protein (or a derivative thereof) with a proteoglycan molecule, followed by detecting any cleavage of the proteoglycan molecule.

V. Development of ADAMTS-8 Inhibitors, Antisense Polynucleotides, and RNAi Sequences

The present invention features identification of ADAMTS-8 inhibitors. A screen assay suitable for this purpose includes contacting an ADAMTS-8 protein (or a derivative thereof) with a proteoglycan substrate in the presence or absence of a compound of interest. A proteolytic activity of the ADAMTS-8 protein (or its derivative) is evaluated in the presence or absence of the compound to determine if the compound has any inhibitory effect on the proteolytic activity. See, for example, Hashimoto, et al., supra. High throughput screening assays or compound libraries can be employed to facilitate the identification of ADAMTS-8 inhibitors. ADAMTS-8 enhancers can be similarly identified.

ADAMTS-8 inhibitors can also be identified using three-dimensional structural analysis or computer aided drug design. The latter method entails determination of binding sites for inhibitors based on the three-dimensional structures of ADAMTS-8 proteins and their proteoglycan substrates (e.g., aggrecan). Molecules reactive with the binding site(s) on ADAMTS-8 or its substrate are selected. Candidate molecules are then assayed for determining any inhibitory effect. Other methods that are suitable for developing protease inhibitors can also be used for the identification of ADAMTS-8 inhibitors.

ADAMTS-8 inhibitors can be, for example, proteins, peptides, antibodies, chemical compounds, or small molecules. In one embodiment, an ADAMTS-8 inhibitor identified by the present invention can inhibit at least 20%, 30%, 40%, 50%, 60%, 70%, 80%, 90%, or more of a proteolytic activity (e.g., aggrecanase activity) of an ADAMTS-8 protein. In another embodiment, an ADAMTS-8 inhibitor identified by the present invention can specifically inhibit a proteolytic activity of an ADAMTS-8 protein but not other non-ADAMTS proteases, such as MMPs. In yet another embodiment, an ADAMTS-8 inhibitor identified by the present invention can specifically inhibit a proteolytic activity of an ADAMTS-8 protein but not other ADAMTS family members. By “specifically inhibit,” it means that an inhibitor can reduce or eliminate an activity of the target protein, but does not significantly affect the activities of other proteins. In some examples, inhibitors specific for ADAMTS-8 proteins inhibit less than 10%, 5%, or 1% of the activities of other proteases. In some other examples, inhibitors specific for ADAMTS-8 proteins have no detectable effect on other proteases.

ADAMTS-8 inhibitors of the present invention can be used to determine the presence or absence of, or to quantitate, ADAMTS-8 proteins in a sample. By correlating the presence or the expression level of ADAMTS-8 proteins with a disease, one of skill in the art can use ADAMTS-8 proteins as biological markers for the diagnosis of the disease or determining its severity.

Where ADAMTS-8 inhibitors are intended for diagnostic purposes, it may be desirable to modify the inhibitors, for example, with a ligand group (e.g., biotin or other molecules having specific binding partners) or a detectable marker group (e.g., a fluorophore, a chromophore, a radioactive atom, an electron-dense reagent, or an enzyme). Molecules having specific binding partners include, but are not limited to, biotin and avidin or streptavidin, IgG and protein A, and numerous receptor-ligand couples known in the art. Enzyme markers that are conjugated to ADAMTS-8 inhibitors can be detected by their enzymatic activities. For example, horseradish peroxidase can be detected by its ability to convert tetramethylbenzidine (TMB) to a blue pigment, which is quantifiable by a spectrophotometer.

The present invention also features polynucleotides that are antisense to ADAMTS-8 sequences. An antisense polynucleotide can form hydrogen bonds to the sense polynucleotide that encodes an ADAMTS-8 protein. An antisense polynucleotide can be complementary to a coding or non-coding region of an ADAMTS-8 sequence. An antisense polynucleotide can be complementary to the entire strand of an ADAMTS-8 transcript or to only a portion thereof. An antisense polynucleotide can include, without limitation, about 5, 10, 15, 20, 25, 30, 35, 40, 45, 50, or more nucleotide residues.

Any method known in the art can be used for preparing antisense polynucleotides. In one embodiment, antisense polynucleotides are chemically synthesized using naturally occurring nucleotides. In another embodiment, antisense polynucleotides are synthesized using modified nucleotides to increase the biological stability of the molecules or the physical stability of the duplex formed between the antisense and sense polynucleotides. Examples of modified nucleotides include, but are not limited to, phosphorothioate derivatives, acridine substituted nucleotides, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladen4exine, unacil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, (acp3)w, and 2,6-diaminopurine. Antisense polynucleotides can also be prepared using both naturally occurring and modified nucleotides.

In yet another embodiment, antisense polynucleotides are produced biologically using expression vectors. These expression vectors encode polynucleotides in an orientation such that RNA transcribed therefrom is of an antisense orientation to the target polynucleotides.

In another embodiment, the antisense molecules are α-anomeric polynucleotide molecules. α-anomeric polynucleotide molecules can form specific double-stranded hybrids with complementary RNA in which, contrary to the usual β-units, the strands run parallel to each other. In still yet another embodiment, the antisense molecules include 2′-o-methylribonucleotides or chimeric RNA-DNA analogues.

In yet another embodiment, the antisense molecules are ribozymes. Ribozymes are catalytic RNA molecules which can cleave single-stranded polynucleotides (e.g., mRNA) to which they have a complementary region. Ribozymes specific for ADAMTS-8 RNA can be designed or selected using various methods known in the art.

In a further embodiment, the antisense molecules are capable of forming a triple helical structure with a regulatory region of the ADAMTS-8 gene, thereby preventing the transcription of the ADAMTS-8 gene.

Antisense polynucleotides are typically administered to a subject in pharmaceutical compositions, or generated in situ from expression vectors. In one example, antisense polynucleotides are directly injected at a tissue site (e.g., articular cartilage). In another example, antisense polynucleotides are administered systemically. For systemic administration, antisense molecules can be first modified such that they can specifically bind to receptors or antigens expressed on the surface of a selected cell. Expression vectors that encode antisense molecules can be administered to a tissue site by any conventional means. To achieve sufficient intracellular concentrations of the antisense molecules, strong promoters, such as pol II or pol III promoter, can be used in the expression vectors. The directly administered or vector-produced antisense molecules can hybridize or bind to cellular mRNA or genomic DNA, thereby inhibiting the translation or transcription of ADAMTS-8 proteins.

The present invention further contemplates the use of RNA interference (“RNAi”) to inhibit the expression of ADAMTS-8 proteins. RNAi provides a mechanism of gene silencing at the mRNA level. The RNAi sequences of the present invention can have any desired length. In many instances, the RNAi sequences have at least 10, 15, 20, 25, or more consecutive nucleotides. The RNAi sequences can be dsRNA or other types of polynucleotides, provided that they can form a functional silencing complex to degrade the target mRNA transcript.

In one embodiment, the RNAi sequences of the present invention comprise or consist of a short interfering RNA (siRNA). In many applications, the siRNA are dsRNA having about 19-25 nucleotides. siRNAs can be produced endogenously by degradation of longer dsRNA molecules by an RNase III-related nuclease Dicer. siRNAs can also be introduced into cells exogenously or by transcription from expression vectors. Once produced, siRNAs assemble with protein components to form endoribonuclease-containing complexes known as RNA-induced silencing complexes (RISCs). Activated RISCs cleave and destroy complementary mRNA transcripts. This sequence-specific mRNA degradation results in gene silencing.

At least two methods can be employed to achieve siRNA-mediated gene silencing. In the first method, siRNAs are synthesized in vitro and then introduced into cells to transiently suppress gene expression. Synthetic siRNAs provide an easy and efficient way to achieve RNAi. In many embodiments, the siRNAs are duplexes of short mixed oligonucleotides which include about 19-23 nucleotides with symmetric dinucleotide 3′ overhangs (e.g., UU or dTdT 3′ overhangs). These siRNAs can specifically suppress targeted gene translation in mammalian cells without activation of DNA-dependent protein kinase (PKR). Activation of PKR has been reported to cause non-specific repression of translation of many proteins.

In the second method, siRNAs are expressed from vectors. This approach can be used to stably or transiently express siRNAs in cells or transgenic animals. In one embodiment, siRNA expression vectors are engineered to drive siRNA transcription from polymerase III (pol III) transcription units. In many instances, Pol III transcription units employ a short AT rich transcription termination site that leads to the addition of 2 bp overhangs (e.g., UU) to hairpin siRNAs—a feature that is helpful for siRNA function. The Pol III expression vectors can also be used to create transgenic animals that express siRNAs. In addition, tissue specific promoters can be used to express siRNAs in selected cells or tissues. A similar approach can be employed to create tissue-specific knockdown animals. In another embodiment, long double-stranded RNAs (dsRNAs) are first expressed from a vector. The long dsRNAs are then processed into siRNAs by Dicer to generate gene-specific silencing.

Numerous 3′ dinucleotide overhangs (e.g., UU) can be used for siRNA design. In some cases, G residues in the overhang are avoided to reduce the risk of the siRNA being cleaved by RNase at the single-stranded G residues.

In one embodiment, the siRNAs of the present invention has about 30-50% GC content. In another embodiment, stretches of over 4 consecutive Ts or As in the target sequence are avoided when designing siRNAs to be expressed from an RNA pol III promoter. In yet another embodiment, siRNAs are selected such that the target mRNA sequence is not highly structured or bound by regulatory proteins. In still another embodiment, the potential target sites are compared to the appropriate genome database. Target sequences with more than 16-17 contiguous base pairs of homology to other coding sequences may be eliminated from consideration.

In still yet another embodiment, siRNAs are designed to have two inverted repeats separated by a short spacer sequence and end with a string of Ts that serve as a transcription termination site. This design produces an RNA transcript that is predicted to fold into a short hairpin siRNA. The selection of siRNA target sequence, the length of the inverted repeats that encode the stem of a putative hairpin, the order of the inverted repeats, the length and composition of the spacer sequence that encodes the loop of the hairpin, and the presence or absence of 5′-overhangs, can vary to achieve desired results.

In another embodiment, the hairpin siRNA expression cassette is constructed to contain the sense strand of the target, followed by a short spacer, the antisense strand of the target, and 5-6 Ts as transcription terminator. The order of the sense and antisense strands within the siRNA expression constructs can be altered without affecting the gene silencing activities of the hairpin siRNA. In some instances, however, the reversal of the order may cause partial reduction in gene silencing activities.

In yet another embodiment, the length of the nucleotide sequence being used as the stem of an siRNA expression cassette ranges from about 19 to 29. The loop size can range from 3 to 23 nucleotides. Other stem lengths or loop sizes can also be used.

A variety of methods are available for selecting siRNA targets. In one example, the siRNA targets are selected by scanning an mRNA sequence for AA dinucleotides and recording the 19 nucleotides immediately downstream of the AA. In another example, the selection of the siRNA target sequences is purely empirically determined, provided that the target sequence starts with GG and does not share significant sequence homology with other genes as analyzed by BLAST search. In still another example, the selection of the siRNA target sequences is based on the observation that accessible sites in endogenous mRNA can be targeted for degradation by synthetic oligodeoxyribonucleotide/RNase H method (Lee, et al., NATURE BIOTECHNOLOGY, 20:500-505 (2002)).

In one embodiment, the target sequences for RNAi are 21-mer sequence fragments selected based on ADAMTS-8 coding sequences. The 5′ end of each target sequence includes dinucleotide “NA,” where “N” can be any base and “A” represents adenine. The remaining 19-mer sequence has a GC content of between 35% and 55%. In addition, the remaining 19-mer sequence does not include any four consecutive A or T (i.e., AAAA or TTTT), three consecutive G or C (i.e., GGG or CCC), or seven “GC” in a row.

Additional criteria can also be included for RNAi target sequence design. For instance, the GC content of the remaining 19-mer sequence can be limited to between 45% and 55%. Moreover, any 19-mer sequence having three consecutive identical bases (i.e., GGG, CCC, TTT, or AAA) or a palindrome sequence with 5 or more bases can be excluded. Furthermore, the remaining 19-mer sequence can be selected to have low sequence homology to other genes. In one example, potential target sequences are searched by BLASTN against NCBI's human UniGene cluster sequence database. The human UniGene database contains non-redundant sets of gene-oriented clusters. Each UniGene cluster includes sequences that represent a unique gene. 19-mer sequences that produce no hit to other human genes under the BLASTN search can be selected. During the search, the e-value may be set at a stringent value (such as at “1”).

The effectiveness of the siRNA sequences of the present invention can be evaluated using numerous methods. For instance, an siRNA sequence of the present invention can be introduced into a cell which expresses ADAMTS-8. The polypeptide or mRNA level of ADAMTS-8 in the cell can be detected. A decrease in the ADAMTS-8 expression level after the introduction of the siRNA sequence indicates that the siRNA sequence introduced is effective for inducing RNA interference.

The expression levels of other genes can also be monitored before and after the introduction of siRNA sequences. siRNA sequences that have inhibitory effect on the expression of the ADAMTS-gene 8 but not other genes can be selected. In addition, different siRNA sequences can be introduced into the same cell for the suppression of the ADAMTS-8 gene.

VI. Disease Treatment

The present invention features the use of ADAMTS-8 modulators to treat protease-related diseases. ADAMTS-8 modulators include, but are not limited to, ADAMTS-8 antibodies, ADAMTS-8 inhibitors, ADAMTS-8 antisense or RNAi sequences, and vectors encoding or comprising ADAMTS-8 antisense or RNAi sequences. Protease-related diseases that are amenable to the present invention include, without limitation, cancer, inflammatory joint disease, osteoarthritis, rheumatoid arthritis, septic arthritis, periodontal diseases, corneal ulceration, proteinuria, coronary thrombosis from atherosclerotic plaque rupture, aneurysmal aortic disease, inflammatory bowel disease, Crohn's disease, emphysema, acute respiratory distress syndrome, asthma, chronic obstructive pulmonary disease, Alzheimer's disease, brain and hematopoietic malignancies, osteoporosis, Parkinson's disease, migraine, depression, peripheral neuropathy, Huntington's disease, multiple sclerosis, ocular angiogenesis, macular degeneration, aortic aneurysm myocardial infarction, autoimmune disorders, degenerative cartilage loss following traumatic joint injury, head trauma, dystrophobic epidermolysis bullosa, spinal cord injury, acute and chronic neurodegenerative diseases, osteopenias, tempero mandibular joint disease, demyelating diseases of the nervous system, organ transplant toxicity and rejection, cachexia, allergy, tissue ulcerations, restenosis, and other diseases characterized by abnormal degradation of extracellular matrix proteins or proteoglycan molecules.

Treatment can include both therapeutic treatments and prophylactic or preventative measures. Those in need of treatment include individuals already having a particular medical disorder, as well as those who may ultimately acquire the disorder. In many examples, a desired treatment regulates the proteolytic activity or gene expression of ADAMTS-8 so as to prevent or ameliorate clinical symptoms of the disease. ADAMTS-8 modulators can function, for example, by preventing the interaction between ADAMTS-8 and its proteoglycan substrate, reducing or eliminating the catalytic activity of ADAMTS-8, or reducing or eliminating the transcription or translation of the ADAMTS-8 gene.

In one embodiment, ADAMTS-8 modulators (e.g., antibodies or inhibitors) are administered to humans or animals in pharmaceutical compositions. A pharmaceutical composition typically includes a pharmaceutically acceptable carrier and a therapeutically effective amount of an ADAMTS-8 modulator. Examples of pharmaceutically acceptable carriers include solvents, solubilizers, fillers, stabilizers, binders, absorbents, bases, buffering agents, lubricants, controlled release vehicles, diluents, emulsifying agents, humectants, lubricants, dispersion media, coatings, antibacterial or antifungal agents, isotonic and absorption delaying agents, and the like, that are compatible with pharmaceutical administration. The use of carrier media and agents for pharmaceutically active substances is well-known in the art. Supplementary agents can also be incorporated into the compositions.

The pharmaceutical compositions of the present invention can be formulated to be compatible with its intended route of administration. Examples of routes of administration include parenteral, intravenous, intradermal, subcutaneous, oral, inhalation, transdermal, rectal, transmucosal, topical, and systemic administration. In one example, the administration is carried out by using an implant.

In one embodiment, solutions or suspensions used for parenteral, intradermal, or subcutaneous applications include the following components: a sterile diluent such as water, saline solution, fixed oils, polyethylene glycols, glycerine, propylene glycol, or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates, or phosphates; and agents for the adjustment of tonicity such as sodium chloride or dextrose. The pH of a pharmaceutical composition can be adjusted with acids or bases, such as hydrochloric acid or sodium hydroxide. In one example, parenteral preparations are enclosed in ampoules, disposable syringes, or multiple dose vials made of glass or plastic.

A pharmaceutical composition of the present invention can be administered to a patient or animal such that the ADAMTS-8 modulator comprised therein is in a sufficient amount to reduce or abolish the targeted ADAMTS-8 activity or expression. Suitable therapeutic dosages for an ADAMTS-8 antibody or inhibitor can range, without limitation, from 5 mg to 100 mg, from 15 mg to 85 mg, from 30 mg to 70 mg, or from 40 mg to 60 mg. Dosages below 5 mg or above 100 mg can also be used. ADAMTS-8 antibodies or inhibitors can be administered in one dose or multiple doses. The doses can be administered at intervals such as, without limitation, once daily, once weekly, or once monthly. Dosage schedules for administration of an ADAMTS-8 antibody or inhibitor can be adjusted based on, for example, the affinity of the antibody/inhibitor for its target, the half-life of the antibody/inhibitor, and the severity of the patient's condition. In one embodiment, antibodies or inhibitors are administered as a bolus dose, to maximize their circulating levels. In another embodiment, continuous infusions are used after the bolus dose.

Toxicity and therapeutic efficacy of ADAMTS-8 modulators can be determined by standard pharmaceutical procedures in cell culture or experimental animal models. For instance, the LD₅₀ (the dose lethal to 50% of the population) and the ED₅₀ (the dose therapeutically effective in 50% of the population) can be determined. The dose ratio between toxic and therapeutic effects is the therapeutic index, and can be expressed as the ratio LD₅₀/ED₅₀. In one example, modulators which exhibit large therapeutic indices are selected.

The data obtained from cell culture assays or animal studies can be used in formulating a range of dosages for use in humans. In many cases, the dosage of such compounds or modulators may lie within a range of circulating concentrations that exhibit an ED₅₀ with little or no toxicity. The dosage may vary within this range depending upon the dosage form employed and the route of administration utilized. For any modulator used according to the present invention, a therapeutically effective dose can be estimated initially from cell culture assays or animal models. In one embodiment, a dose may be formulated in animal models to achieve a circulating plasma concentration range that exhibits an IC₅₀ (i.e., the concentration of the test inhibitor which achieves a half-maximal inhibition of symptoms) as determined by cell culture assays. Levels in plasma may be measured, for example, by high performance liquid chromatography. The effects of any particular dosage can be monitored by suitable bioassays. Examples of bioassays include DNA replication assays, transcription-based assays, GDF protein/receptor binding assays, creatine kinase assays, assays based on the differentiation of pre-adipocytes, assays based on glucose uptake in adipocytes, and immunological assays.

The dosage regimen for administration of a pharmaceutical composition of the present invention can be determined by the attending physician based on various factors such as the site of pathology, the severity of disease, the patient's age, sex, and diet, the severity of any inflammation, time of administration, and other clinical factors. In certain embodiments, systemic or injectable administration is initiated at a dose which is minimally effective, and the dose will be increased over a preselected time course until a positive effect is observed. Subsequently, incremental increases in dosage will be made limiting to levels that produce a corresponding increase in effect while taking into account any adverse affects that may appear. The addition of other known factors to a final composition may also affect the dosage.

The present invention also contemplates treatment of diseases that are caused by or associated with abnormal accumulation of aggrecan or other proteoglycans. In one embodiment, the treatment includes administering a pharmaceutical composition comprising an ADAMTS-8 protein or a functional derivative thereof to a human or animal affected by such a disease. In another embodiment, vector-based therapies are used to correct the abnormal accumulation of proteoglycans. These therapies typically comprise introducing an expression vector or a gene-delivery vector that encodes an ADAMTS-8 protein or a functional derivative thereof into a human or animal in need thereof.

It should be understood that the above-described embodiments and the following examples are given by way of illustration, not limitation. Various changes and modifications within the scope of the present invention will become apparent to those skilled in the art from the present description.

### EXAMPLES

### Example 1

### Generation of the Phylogram

The following human ADAMTS family member proteins were collected for the generation of a phylogram: ADAMTS-1/AB037767, ADAMTS-2/AJ003125 (with the following changes in the published sequence compared to the sequence used in the phylogram: W643C, P1001L, and S1089C), ADAMTS-3/AF247668, ADAMTS-4/AF148213, ADAMTS-5/AF142099, ADAMTS-6/“SEQ ID NO:2” in US patent application publication 20020120113, ADAMTS-7/AF140675, ADAMTS-8/AF060153 (with the following changes in the published sequence compared to the sequence used in the phylogram: L11P, F13L, L21P, P23Δ, L24Δ, and L129Q, where Δ refers to deletion), ADAMTS-9/AF261918 (with the following changes in the published sequence compared to the sequence used in the phylogram: G46S, and S96T), ADAMTS-10/“SEQ ID NO:9” in PCT publication number WO 02/60942 (with the following change in the published sequence compared to the sequence used in the phylogram: V267I), ADAMTS-12/AJ250725, ADAMTS-13/AJ305314, ADAMTS-14/AF358666 (with the following change in the published sequence compared to the sequence used in the phylogram: L937M), ADAMTS-15/AJ315733, ADAMTS-16/“SEQ ID Nb:4” in PCT publication number WO 02/31163, ADAMTS-17/AJ315735 (with the following changes in the published sequence compared to the sequence used in the phylogram: replacement of amino acid sequence 713ALKD716 with amino acid sequence 713GYIEAAVIPAGARRIRVVEDKPAHSFLALKD743 (SEQ ID NO:1)), ADAMTS-18/AJ311903, ADAMTS-19/AJ311904, and ADAMTS-20/“SEQ ID NO:57” in PCT publication number WO 01/83782. The 19 protein sequence files were concatenated into a single multi-FASTA file and used as input into CLUSTALW 1.81 (see, e.g., the website at www.ebi.ac.uk) and run on IRIX64. CLUSTALW was run under the default settings. The resulting .dnd treefile was used as input for TREEVIEW 1.6.6 (Page, COMPUT. APPL. BIOSCI., 12:357-358 (1996); and the website at taxonomy.zoology.gla.ac.uk/rod/treeview.html) to generate the phylogram.

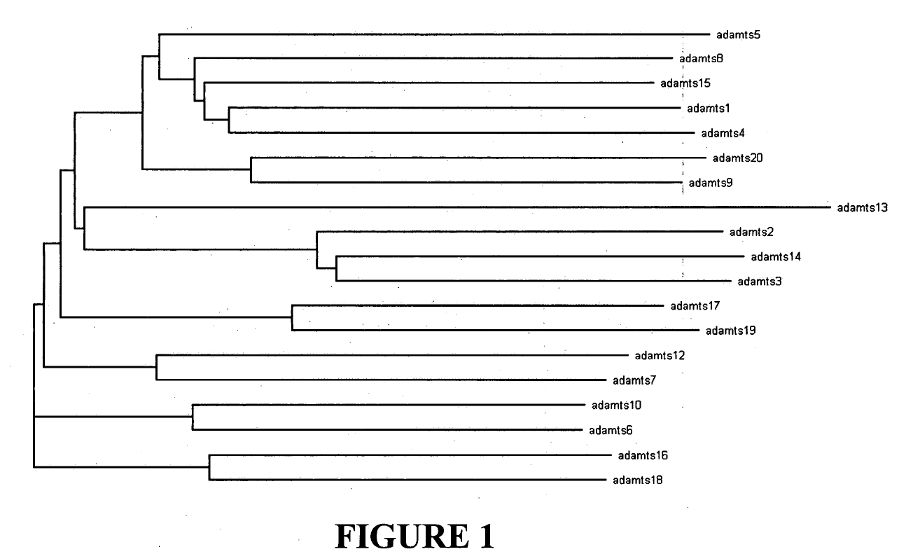

The phylogenetic tree of ADAMTS family members are shown in FIG. 1. The phylogram groups the proteins together based upon sequence relatedness. ADAMTS family members that were grouped together by the program were compared to the known functional information for ADAMTS family members that have been characterized. For instance, ADAMTS-2, 3 and 14 are predicted to be pro-collagen processing enzymes. These family members are most similar to each other by sequence homology and form a unique cluster on the phylogenetic tree. For another instance, mutations in ADAMTS-13 have been shown to cause defects in vWF processing resulting in thrombotic thrombocytopenic purpura. This family member forms its own node on the phylogenetic tree. In addition, ADAMTS-1, 4, 5, and 9 have been shown to cleave aggrecan with varying efficiency. Analysis of sequence homology demonstrated a cluster that contained all of these aggrecan-degrading ADAMTSs plus ADAMTS-8, 15, and 20, suggesting that ADAMTS-8 may also possess aggrecan-cleavage activities. ADAMTS-8 was subsequently cloned, expressed, and purified to determine its ability to cleave aggrecan.

To date, at least 19 members of the ADAMTS family have been identified. Less than half of the ADAMTS proteins have had functions ascribed to them, leaving at least 10 members that have no known function. Construction of a phylogenetic tree (FIG. 1) based upon sequence similarities between family members led to the observation that those ADAMTS family members with similar functions (e.g. demonstrated aggrecan-degrading activity or procollagen processing activity) were grouped together. This suggested that other members of the putative “aggrecan-degrading” node of the phylogenetic tree may possess significant aggrecanase activity, and perhaps may show greater disease-association to osteoarthritis than ADAMTS-4 or ADAMTS-5. As demonstrated in the following examples, ADAMTS-8, another member of the “aggrecan-degrading” node, is capable of cleaving aggrecan at the osteoarthritis-relevant Glu³⁷³-Ala³⁷⁴ bond and therefore the structure/function association predicted by sequence homologies holds true for this protein.

### Example 2

### Construction of an ADAMTS-8 Expression Vector

The DNA sequence for ADAMTS-8 was deposited in GenBank by Vázquez et al., supra (accession number AF060153). For gene isolation, 4 sets of oligonucleotide primer pairs that span the ADAMTS-8 open reading frame were designed:

The first primer pair includes ATGTTCCCCGCCCCCGCCGCC CCCCGGTG (SEQ ID NO:2) and GGATCCCCCGAGGCGCTCGATCTTGAACT (SEQ ID NO:3). The second primer pair includes GGATCCGGCCGGGCGACCGGGGGC (SEQ ID NO:4) and CTCTAGAAGCTCTGTGAGATACATGGCGCT (SEQ ID NO:5). The third primer pair includes CTCTAGACGGCGGGCACGGAGACTGTCTCCTG GATGCCCCTGGTGCGGCCCTGCCCCTCCCCACA (SEQ ID NO:6) and ACGTGT ATTTGACTTTTGGGGGGAAGACCTCGCCAGGGACTGTCAGGAGCTGCACTGTCAG AGGCTC (SEQ ID NO:7). The fourth primer pair includes CACACGTTCTTTGTTC CTAATGACGTGGACTTTAG (SEQ ID NO:8) and GCGGCCGCTCACAGGGG GCACAGCTGGCTTTC (SEQ ID NO:9).

PCR amplification was performed on an adult lung cDNA library using the GC kit from Clontech following the manufacturer's recommendations. Amplification of the PCR products was performed in a Perkin Elmer 9600. Fifty microliter PCR reactions were heated to 95° C. for a 1 minute pre-incubation step immediately followed by 25 cycles consisting of incubation at 95° C. for 15 seconds followed by incubation at 68° C. for 2 minutes. The resulting PCR products were purified, digested with appropriate restriction enzymes (EcoR I/BamH I, BamH I/Xba I, Xba I/Afl III, Afl III/Not I respectively), and ligated together into the CHO expression vector pHTop (a derivative of pED). The PCR insert was verified by DNA sequencing.

The ADAMTS-8 expression construct was modified by addition of a Strep-tag® sequence (IBA). The tag was added using PCR primers with a 3′ extension encoding a five amino acid linker (GSGSA (SEQ ID NO:10)) followed by additional sequence encoding an 8 amino acid Strep-tag (WSHPQFEK (SEQ ID NO:11)). These 13 amino acids were added as a C-terminal translational fusion to the final amino acid of the ADAMTS-8 open reading frame. The PCR primer pair consisted of a forward primer CTTCTAGACGGCGGGCACGGAGAC (SEQ ID NO:12) and a reverse primer TTCTAGAGCGGCCGCCTTATTTTTCGAACTGCGGGTGGCTCCAAGCAGATCCGGA TCCCAGGGGGCATAGCTGGCTTTCGCA (SEQ ID NO:13). Amplification of the PCR product was performed in a Perkin Elmer 9600. Pfu Turbo Hotstart (Stratagene) was used as the DNA polymerase and the reaction conditions followed those recommended by the manufacturer. PCR reactions were initially heated to 94° C. for 2 minutes, followed by 25 cycles of 94° C. for 15 seconds/70° C. for 2 minutes. After the final cycle, the PCR reactions were held for 5 minutes at 72° C. The PCR product was purified, digested with the appropriate restriction enzymes (Bgl II/Not I) and then ligated together with the appropriate ADAMTS-8 fragments into the pHTop expression vector.

Several amino acid variations were identified when comparing AF060153 to the cloned sequence. The observed changes were restricted to the signal peptide and the prodomain. Two of the variations in the signal sequence of the ADAMTS-8 isolate were also found in a GenBank database sequence submission, accession number AAB74946. The observed changes in the ADAMTS-8 isolate that could not be ascribed to allelic variations (e.g., F13 and F14 deleted and L129Q) resulted in a 25 amino acid signal peptide and a single amino acid change in the prodomain. These changes did not affect expression or activity of the mature protein by virtue of their locations and were left unchanged in the expression construct. The predicted protein sequence for the mature portion of the protein was identical to AF060153.

### Example 3

### Establishment of a CHO Cell Line for Expression of ADAMTS-8

CHO/A2 cells were used to establish the ADAMTS-8 expressing stable cell line. The CHO/A2 cell line was derived from CHO DUKX B11 by stable integration of the transcriptional activator tTA, a fusion protein comprised of the Tet repressor and the herpes virus VP16 transcriptional domain. The ADAMTS-8/pHTop expression vector contains six repeats of the tet operator upstream of the ADAMTS-8 sequence. Binding of tTA to the Tet operator in pHTop activates transcription of the downstream gene. The gene encoding dihydrofolate reductase is also contained on the pHTop expression vector, allowing for selection of stable transfectants by virtue of methotrexate resistance. A CHO cell line expressing extracellular ADAMTS-8 was established by transfecting pHTop/ADAMTS-8 DNA into CHO/A2 cells using the manufacturer's recommended protocol for lipofection (Lipofectin from InVitrogen). Clones were selected in 0.02 μM methotrexate. Cell lines expressing the highest level of ADAMTS-8 protein were selected by monitoring ADAMTS-8 antigen in the CHO conditioned media by Western blotting using an anti-Strep-tag antibody conjugated to horseradish peroxidase (HRP) (Southern Biotech) followed by ECL chemiluminescence (Amersham Biosciences) and autoradiography.

### Example 4

### Purification of ADAMTS-8

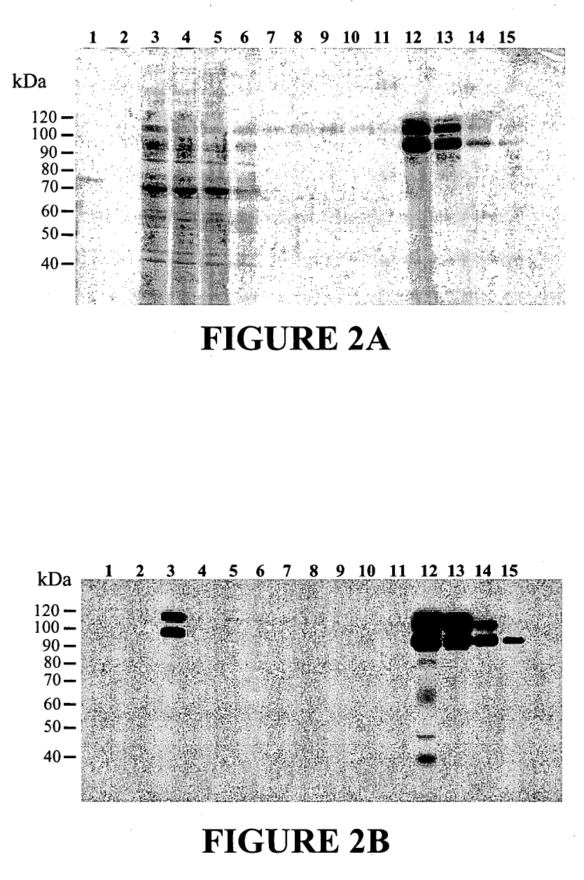

Conditioned medium (300 ml) from a stable CHO cell line expressing ADAMTS-8 was collected and concentrated 3-fold (10 ml) by ultrafiltration using a stir cell (Amicon) fitted with a 10 kDa MWCO (molecular weight cut-off) filter. Avidin immobilized on cross-linked 6% beaded agarose (1 ml) from Sigma was mixed with the concentrated conditioned medium for 1 hour at 4° C. to remove any contaminating biotin. The supernatant was recovered following centrifugation, and loaded onto a 1 ml Strep-Tactin column (IBA). The column was washed with five 1 ml aliquots of Buffer W (100 mM Tris, pH 8.0, 150 mM NaCl), and the bound protein was eluted from the column with Buffer W containing 2.5 mM desthiobiotin (Sigma). Aliquots of concentrated conditioned medium, column flow through, wash and elution fractions were analyzed by 10% SDS-PAGE gel analysis (FIG. 2A) followed by Western analysis using the anti Strep-Tag II polyclonal antiserum (IBA) and ECL detection by autoradiography (FIG. 2B).

FIG. 2A illustrates the 10% SDS-PAGE of protein fractions from Strep-tag purification of ADAMTS-8 from CHO conditioned media. The SDS-PAGE was stained with Coomassie Brilliant Blue. Lane 1 indicates the CHO cell conditioned medium. Lane 2 shows the flow through fraction (filtrate) from ultrafiltration. Lane 3 is the concentrated ultrafiltration retentate fraction. Lane 4 represents Strep-Tactin column flow-through fraction. Lanes 5-9 are Strep-Tactin column wash fractions. Lanes 10-15 depict Strep-Tactin column elution fractions.

FIG. 2B shows a corresponding Western blot of the SDS-PAGE of FIG. 2A. The Western analysis employed the anti Strep-Tag II polyclonal antiserum (IBA).

The expected molecular weights of unprocessed and furin-processed ADAMTS-8 containing the Strep-tag, not accounting for altered mobility due to glycosylation, are 95 kDa and 75 kDa, respectively. The major products of the purification were 2 bands that migrated on SDS-PAGE at apparent molecular weights of 110 kDa and 95 kDa (FIG. 2A, lane 12) and bound the Strep-tag antibody on Western blots (FIG. 2B, lane 12). Co-expression of soluble PACE (Furin or paired basic amino acid cleaving enzyme) with the ADAMTS-8 expression construct in CHO/A2 cells resulted in the elimination of the 110 kDa pro-ADAMTS-8 band with a concomitant increase in the amount of the 95 kDa band, suggesting that the 110 kDa band represented secreted pro-ADAMTS-8. There are 5 putative N-linked glycosylation sites within the mature ADAMTS-8 protein, which presumably accounts for the increased apparent molecular weight from the 75 kDa predicted for mature ADAMTS-8 to the observed 95 kDa. Western analysis of the purified protein fractions showed a preponderance of full-length protein, and only a minor proportion of immunoreactive bands of decreased molecular weight (lane 12 in FIG. 2B). These minor products may be the result of degradation or autocatalysis of the mature ADAMTS-8 protein. An elution fraction containing both the pro-ADAMTS-8 and processed mature ADAMTS-8 was used for subsequent activity analyses.

In this example, the full-length ADAMTS-8 cDNA was appended with a sequence encoding a carboxy-terminal Strep-tag and expressed in CHO cells. The protein was efficiently expressed and secreted to the conditioned medium. The full-length protein accumulated in the conditioned medium and was not appreciably proteolyzed into smaller products. This observation was supported by retention of the carboxy-terminal tag as determined by Western blotting with anti-Strep-tag antibodies and verified by the ability of the most of the protein to bind to Strep-Tactin resin. In contrast, the recombinant ADAMTS-4 as used for comparison was spontaneously proteolyzed at sites within the C-terminal domains, which generated a truncated molecule lacking the spacer domain. Truncation of ADAMTS-4 appears to be an autoproteolytic event, because a modified form of ADAMTS4 in which the catalytic activity has been destroyed by an E362Q active-site mutation did not demonstrate this spontaneous C-terminal truncation (Flannery, et al., J. BIO. CHEM., 277:42775-42780 (2002)). In addition, recombinant ADAMTS-5 (Aggrecanase-2) can self-truncate its C-terminus. Recombinant ADAMTS-12 also displays this characteristic of secondary C-terminal proteolysis (Cal, et al., J. BIOL. CHEM., 276:17932-17940 (2001)), though from the published report it is unclear if it is an autoproteolytic event or if it is mediated by other protease(s). Furthermore, expression of ADAMTS-1 in 293T cells reportedly resulted in three forms of the protein—namely, a p110 form representing pro-ADAMTS-1, a p87 form which is presumed to be full-length mature ADAMTS-1, and a p65 form which constitutes mature ADAMTS-1 C-terminally truncated within the spacer domain (Rodrigues-Manzaneque, et al., J. BIOL. CHEM., 275:33471-33479 (2000)). Consistent with the observations with ADAMTS-4, an ADAMTS-1 active-site mutant did not C-terminally truncate, suggesting that an autoproteolytic mechanism is responsible for removal of the C-terminal domains.

Based on these data, it was surprising that most recombinant ADAMTS-8 isolated in this example retained its C-terminal domains and did not appear to autoproteolyze or become cleaved by another protease. The proteolytic activity of this recombinant ADAMTS-8 protein was verified by using the α-2 macroglobulin binding assay. Accordingly, the carboxy-terminal thrombospondin and spacer domains in ADAMTS-8 are uncharacteristically refractory to secondary processing by either its own catalytic activity or other processing enzymes, therefore providing a unique opportunity to assess the catalytic efficiency of a stable full-length ADAMTS protein.

### Example 5

### Isolation of RNA from Articular Cartilage

Non-osteoarthritic human articular cartilage was obtained from Clinomics (Pittsfield, Mass.), and osteoarthritic human articular cartilage was obtained from New England Baptist Hospital (Boston, Mass.). Samples were flash frozen in liquid nitrogen at the time of collection and stored at −80° C. For RNA isolation, 1 gram of frozen articular cartilage was milled twice (1 minute each, with a 2 minute cooling step between each milling) in a Spex Certiprep freezer mill (model 6750) at 15 Hz under liquid nitrogen. RNA was then isolated according to the method of McKenna et al., ANAL. BIOCHEM., 286:80-85 (2000), with the following modifications. The milled cartilage was suspended in 4 mL of ice-cold 4M guanidinium isothiocyanate (GITC, Gibco-BRL) containing 2.5 μL of 2-mercaptoethanol (2-ME). The suspension was immediately homogenized on ice for 1 minute using a Polytron homogenizer (Kinematica AG) at highest speed. The homogenized cartilage lysate was centrifuged at 1500×g for 10 minutes at 4° C., the supernatant was saved, and the resulting pellet was homogenized again as before in another 4 ml of GITC/2-ME and centrifuged again at 1500×g for 10 minutes at 4° C. The supernatant fractions from each homogenate were combined and 0.65 ml of 25% Triton X-100 (100% stock from Sigma, diluted to 25% in RNase-free dH₂O) was added to the pooled supernatant fractions. After incubation on ice for 15 minutes, 8 ml of RNase-free 3M NaOAc buffer pH 5.5 (Ambion) was added and the solution was incubated for another 15 minutes on ice. The homogenate was then extracted with 15 ml of acid phenol:chloroform 5:1, pH 4.5 (Ambion) by vigorous mixing for 1 minute, incubation on ice for 15 minutes, and centrifugation at 15,000×g for 20 minutes at 4° C. The aqueous phase was then recovered and re-extracted with acid phenol:chloroform using the same procedure as described above. The aqueous phase from the second acid phenol:choloroform extraction was then extracted a third time with 15 ml of phenol:chloroform:IAA 25:24:1 pH 6.7/8.0 (Ambion), mixed vigorously for 1 minute, incubated on ice for 15 minutes, and centrifuged at 15,000×g for 20 minutes at 4° C. The aqueous phase was recovered, and 0.8 volumes of 100% 2-propanol were added. The solution was mixed, incubated on ice for 5 minutes, and centrifuged at 15,000×g for 30 minutes at 4° C. The resulting supernatant was carefully decanted, and the pellet was resuspended in 0.9 ml of buffer RLT+2-ME (Qiagen RNeasy kit). The protocol described in McKenna et al., supra, was then followed to completion from this step onward.

### Example 6

### Tissue Distribution of ADAMTS-8

A human multiple tissue expression array (MTE from Clontech) mRNA dot-blot was probed with a 393 bp ADAMTS-8 fragment which was a BglII/HindIII digested fragment corresponding to base pair 2070 through base pair 2463 of the ADAMTS-8 sequence (Genbank accession number AF060153). The fragment contains a portion of the disintegrin domain and a portion of the central TSP type 1 motif. The fragment sequence was used to query GenBank using the Basic Local Alignment Search Tool, Version 2, from NCBI (NCBI-BlastN). The BlastN search found no significant homology between the ADAMTS-8 probe sequence and other human transcripts in the database, suggesting that the probe fragment would not cross-react with other human transcripts under the MTE hybridization conditions.

The ADAMTS-8 probe fragment was purified and radiolabelled using the Ready-To-Go DNA Labelling Beads (-dCTP) from Amersham Pharmacia Biotech according to the manufacturer's instructions. The radiolabelled fragment was purified away from primers and unincorporated radionucleotides using a Nick column (Amersham Pharmacia Biotech) following the manufacturer's instructions and then used to probe the MTE. Hybridization and subsequent washing conditions for the MTE followed the manufacturer's suggested conditions for a radiolabelled cDNA probe (Clontech MTE Array User Manual).

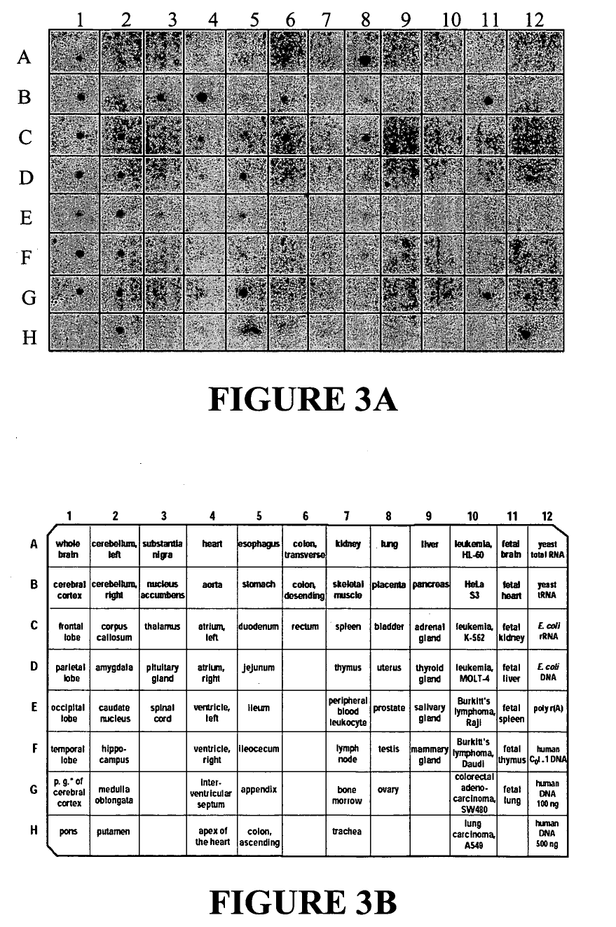

FIG. 3A shows the result of the MTE hybridization analysis using mRNA from 76 different human tissues. A key denoting the placement of mRNA from the different tissues is shown in FIG. 3B. Blank boxes indicate that no mRNA was spotted at those coordinates. The MTE hybridization analysis indicated that ADAMTS-8 has a more narrow tissue distribution and overall lower transcript abundance than the transcripts of the aggrecan-degrading ADAMTS-1 and ADAMTS-4, which have a broad tissue distribution. One of the highest levels of ADAMTS-8 expression was seen in adult lung (FIG. 3, row A, column 8), with lower levels found in fetal lung (FIG. 3, row G, column 11). Expression in adult heart was detectable but low (FIG. 3, column 4), with the exception of aorta that showed a high level of expression (FIG. 3, row B, column 4). Fetal heart (FIG. 3, row B, column 11) showed moderate levels of transcript abundance, and moderate to low level expression was seen in the various subsections of brain, appendix and bladder (e.g., G5, A1-G1, C3-H3, and B3). Various cancer cell lines (FIG. 3, column 10) showed low or no detectable levels of expression.

### Example 7

### Real Time PCR

Tissue expression in human articular cartilage was demonstrated by performing quantitative real-time PCR using TaqMan (Applied Biosystems). The Primer Express program from Applied Biosystems was used to design the following ADAMTS-8 primers and probe: 5P primer GGACCGCTGCAAGTTGTTCT (SEQ ID NO:14), 3P primer GGACACAGATGGCCAGTGTT (SEQ. ID NO:15), and probe CCATCAATCACCTTG GCCTCGAACA (SEQ ID NO:16). The probe for ADAMTS-8 overlapped an exon/intron boundary, making it unable to hybridize to genomic DNA. Primers and a probe were designed to GAPDH and were as follows: 5P primer CCACATCGCTCAGACACCAT (SEQ ID NO:17), 3P primer GCGCCCAATACGACCAAA (SEQ ID NO:18), and probe GGGAAGGTGAAGGTCGGAGTCAACG (SEQ ID NO:19). The TaqMan probes (synthesized by the Wyeth Research Core Technologies Group) contained the 5P-reporter dye 6-FAM and the 3P-quencher TAMRA.

Articular cartilage RNA was isolated from the knee joints of patients that were unaffected by osteoarthritis (disease-free), and from mildly affected and severely affected lesional regions of the knee joints from patients with osteoarthritis. Purified articular cartilage RNA was converted to cDNA prior to real-time PCR by the following protocol, and TaqMan analysis was performed on first-strand cDNA of disease-free and osteoarthritic articular cartilage after reverse transcription of the mRNA. Total RNA (5 μg) was incubated for 10 minutes at 70° C. with 200 pmol of a primer containing a phage T₇ promoter site and a 24 base poly T tail (GGCCAGTGAATTGTAATACGAC TCACTATAGGGAGGCGGTTTTTTTTTTTTTTTTTTTTTTTT (SEQ ID NO:20)). The RNA was then reverse transcribed using 10 Units/μl Superscript II (Invitrogen) in a 20 μl reaction mixture for 1 hour at 50° C. The reaction mixture contained 0.25 μg/μl total RNA, 10 pmol/μl T₇T₂₄ primer, 1×1^{st} Strand Buffer (Invitrogen), 10 mM DTT (Invitrogen), 0.5 mM dNTPs (Invitrogen), and 1 Unit/μl SUPERase-In (Ambion). Following first strand synthesis, second strand synthesis was performed. The reaction mix was brought to a final volume of 150 μl. The reaction contained the first strand mix, and the following reagents (final concentrations)—namely, 1× 2^{nd} Strand Buffer (Invitrogen), 0.2 mM dNTPs (Invitrogen), 0.067 units/μl E. coli DNA Ligase (New England Biolabs), 0.27 units/μl DNA Polymerase I (Invitrogen), and 0.013 units/μl RNase H (Invitrogen). The second strand synthesis reaction was incubated for 2 hours at 16° C. During the last 5 minutes of incubation, T4 DNA Polymerase (Invitrogen) was added to a final concentration of 0.067 units/μl. Following incubation, the reaction was brought to 16.67 mM EDTA and the resulting cDNA was purified using BioMag Carboxyl Terminated beads from PerSeptive Biosystems. The second strand reaction mix was brought to 10% PEG-8000/1.25M NaCl, and added to 10 μl of BioMag beads (pre-washed with 0.5M EDTA). The cDNA and washed BioMag beads were mixed and incubated for 10 minutes at room temperature. The beads were washed 2 times with 300 μl 70% ethanol with the aid of a Magna-Sep magnet from GibcoBRL. The beads were air dried for 2 minutes at room temperature after the final wash. The purified cDNA was eluted from the beads using 10 mM Tris-Acetate (pH 7.8). The eluted cDNA was quantitated by measuring the absorbance of a diluted aliquot of the eluate at 280 nm using a spectrophotometer. Each TaqMan PCR reaction utilized 100 ng of articular cartilage cDNA for the ADAMTS-8 probe/primer set and was performed in duplicate. Expression levels between tissues were normalized using the GAPDH probe/primer set (Applied Biosystems). The reactions components were derived from the TaqMan Universal PCR Master Mix from Applied Biosystems, following manufacturer's instructions, with a final concentration of 900 nmol/μl of primer and 250 nmol/μl probe. Reactions were incubated for 2 minutes at 50° C., followed by 10 minutes at 95° C., and then 40 cycles of 95° C. for 15 seconds and 60° C. for 1 minute. After the final cycle, the reactions were incubated for 2 minutes at 25° C.

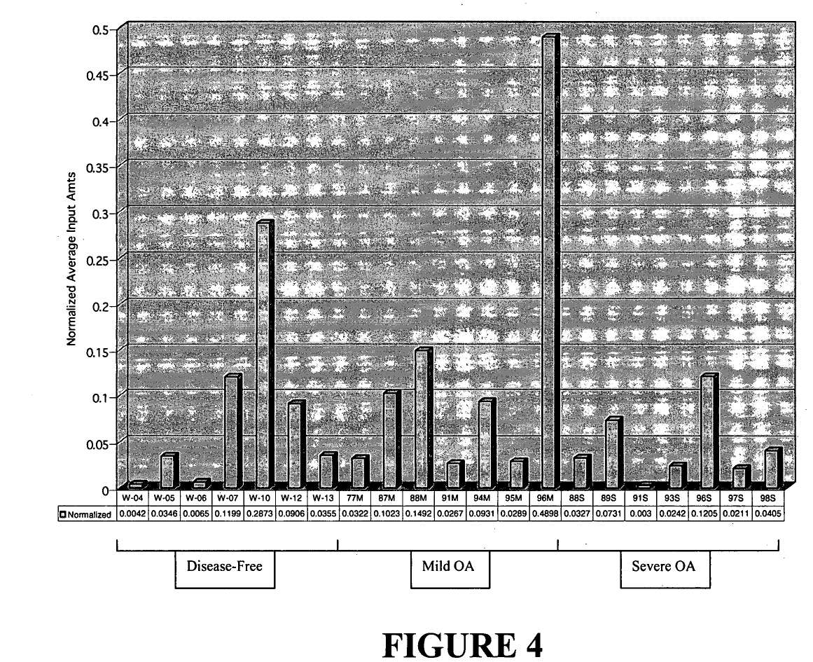

FIG. 4 depicts a histogram of ADAMTS-8 mRNA expression levels in human clinical samples of disease-free and osteoarthritic (OA) cartilage determined by real-time PCR. Samples W-04 through W-13 represent non-OA affected (“Disease-Free”) knee articular cartilage. Samples 77M-96M represent visually unaffected regions of late-stage OA articular cartilage (“Mild OA”). Samples 88S-98S represent severely affected regions of late-stage OA articular cartilage (“Severe OA”). ADAMTS-8 mRNA abundance in each sample was reported as a normalized value, by dividing the averaged data determined for ADAMTS-8 by the averaged data determined for GAPDH in the same sample. The results of the TaqMan analysis showed that there was no significant difference in average transcript level in unaffected cartilage compared to osteoarthritic cartilage, at least in the late-stage OA cartilage that was used in this study. However, the expression level of ADAMTS-8 was significantly increased in the OA cartilage sample 96M. This observation supports for a personalized approach to treat osteoarthritis in selected patients who have elevated ADAMTS-8 expression in their cartilage tissues.

### Example 8

### Production of Monoclonal Antibody AGG-C1 (MAb AGG-C1)

The synthetic peptide CGGPLPRNITEGE (peptide aggc1, SEQ ID NO:21) was coupled to the carrier protein KLH, and the conjugate was used as the immunogen for the production of monoclonal antibodies by standard hybridoma technology. Briefly, BALB/c mice were immunized subcutaneously with 20 μg of immunogen in complete Freund's adjuvant. The injection was repeated twice (biweekly) using peptide in incomplete Freund's adjuvant. Test bleeds were done on the immunized mice, and serum was evaluated by ELISA for reactivity against both the immunizing peptide and ADAMTS-4-digested bovine articular cartilage aggrecan (Flannery, et al., supra). Three days prior to hybridoma fusion, a final immunization without adjuvant was given to the mouse exhibiting highest antibody titer. Spleen cells from this mouse were isolated and fused with FO myeloma cells (American Type Culture Collection, Manassas, Va.) and cultured in HAT selection medium (Sigma-Aldrich, St. Louis, Mo.). Hybridoma culture supernatants were screened against KLH-CGGPLPRNITEGE antigens by ELISA, and against ADAMTS-4-digested aggrecan by Western blotting. Positive hybridoma clones were selected for subcloning by limiting dilution. A single hybridoma cell line, designated AGG-C1, was expanded in culture. Antibody isotype was determined to be IgG1 (κ light chain) using the Mouse Monoclonal Antibody Isotyping kit (Roche, Indianapolis, Ind.) and IgG from 1 liter of culture media was purified by Protein A affinity chromatography.

### Example 9

### Competitive Inhibition ELISA Assays

Competitive inhibition ELISA experiments were performed to demonstrate that MAb AGG-C1 specifically recognized the appropriate aggrecan neoepitope. Streptavidin-coated microtiter plates (Pierce, Rockford, Ill.) were coated with N-terminally biotinylated peptide aggc1 (b-aggc1) by incubating each well with 100 μl of b-aggc1 (100 ng/ml) for 1 h at room temperature. After washing 4 times with phosphate-buffered saline containing 0.01% Tween-20 (PBS-Tween), wells were blocked for 1 h at room temperature with 100 μl of PBS-Tween containing 2% BSA, followed by 4 washes with PBS-Tween.

In order to validate the neoepitope nature of MAb AGG-C1, competition mixtures (100 μl) comprised of MAb AGG-C1 (0.04 μg/ml) and 1.0-1000 nmol/ml of the synthetic peptides GGLPLPRNITEGE (SEQ ID NO:22), GGLPLPRNITEGE ARGSVILTVK-CONH₂ (SEQ ID NO:23), undigested aggrecan, or ADAMTS-4 digested aggrecan were preincubated for 1 h at room temperature. Mixtures were then transferred to b-aggc1 coated wells. After a further incubation for 1 h at room temperature, the plates were washed 4 times with PBS-Tween then incubated for 1 h at room temperature with 100 μl of peroxidase-conjugated secondary goat anti-mouse IgG (1:10,000). Following 4 final washes with PBS-Tween, the wells were incubated with TMB 1 component microwell peroxidase substrate (BioFX Laboratories, Owings Mills, Md.). Color development was terminated by the addition of 0.18 M H₂SO₄, and the absorbance was monitored spectrophotometrically at 450 nm.

For the generation of a standard curve, bovine aggrecan (25 μg in 50 μl) was digested with ADAMTS-4 (0.001 ng-5 ng) for 16 h at 37° C. MAb AGG-C1 was then added to each digest (final antibody concentration of 0.04 μg/ml) and these mixtures were preincubated for 1 h at room temperature, followed by transfer to b-aggc1 coated plates and completion of the ELISA.

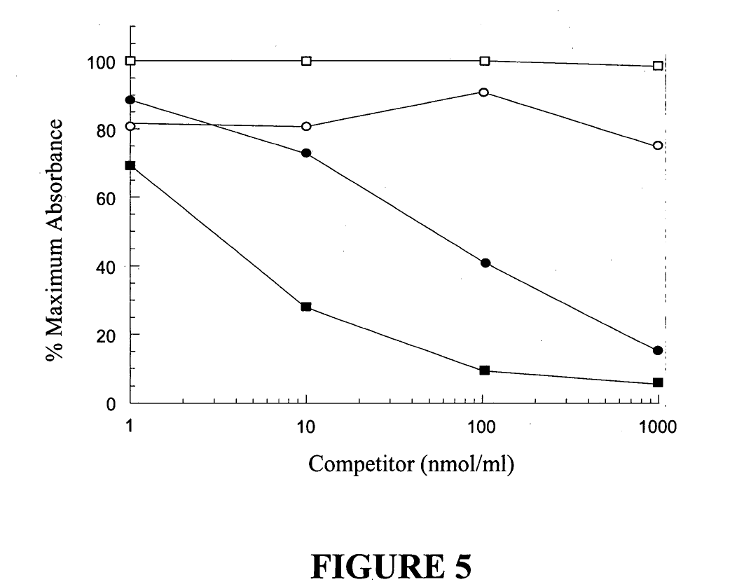

FIG. 5 shows the results of competitive inhibition ELISAs using MAb AGG-C1. Dose-dependent competition was observed for the synthetic peptide GGLPLPRNITEGE (SEQ ID NO:22, the C-terminus of which corresponds to E³⁷³ of aggrecan core protein) and with ADAMTS4 digested aggrecan (closed squares and closed circles, respectively). The synthetic peptide GGPLPRNITEGEARGSVILTVK (SEQ ID NO:23) and undigested aggrecan did not compete in the assay (open squares and open circles, respectively).

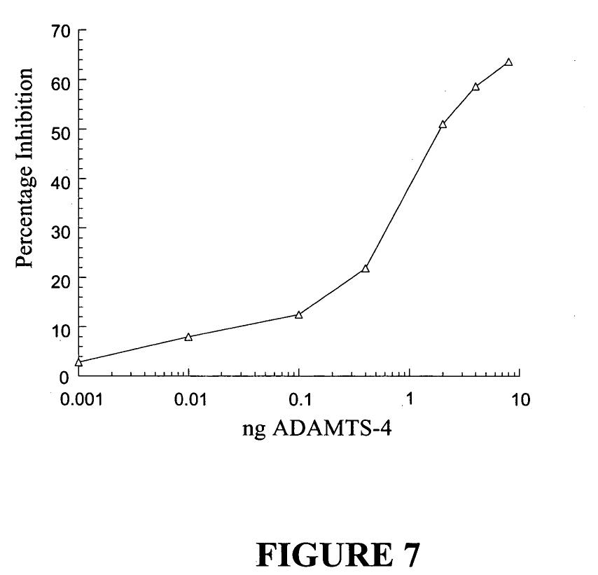

FIG. 7 shows another competitive inhibition ELISA for aggrecanase activity. The standard curve was generated by incubating bovine aggrecan with increasing amounts of recombinant ADAMTS-4 for 16 h at 37° C. followed by addition of MAb AGG-C1 to each digest. Similar assays were performed to estimate the relative aggrecanase activity of ADAMTS-8. Where 0.0135 pM of ADAMTS-4 were required to generate 45% inhibition in the competitive inhibition ELISA, 46.6±4.8 pM of ADAMTS-8 were required to attain a similar level of activity.

### Example 10

### Western Blotting of Aggrecan Digested with ADAMTS-8 and ADAMTS-4

The ability of ADAMTS-8 to cleave aggrecan at the aggrecanase cleavage site (Glu³⁷³-Ala³⁷⁴) that defines osteoarthritis-associated aggrecanase activity was demonstrated using two different monoclonal antibodies—namely, MAb BC-3 and MAb AGG-C1. MAb BC-3 specifically detects the neoepitope N-terminal sequence ³⁷⁴ARGXX . . . (SEQ ID NO:24). MAb AGG-C1 specifically detects the neoepitope C-terminal sequence . . . NITEGE³⁷³ (SEQ ID NO:25). Both neoepitopes are generated by aggrecanase cleavage of the Glu³⁷³-Ala³⁷⁴ peptide bond within the aggrecan interglobular domain.

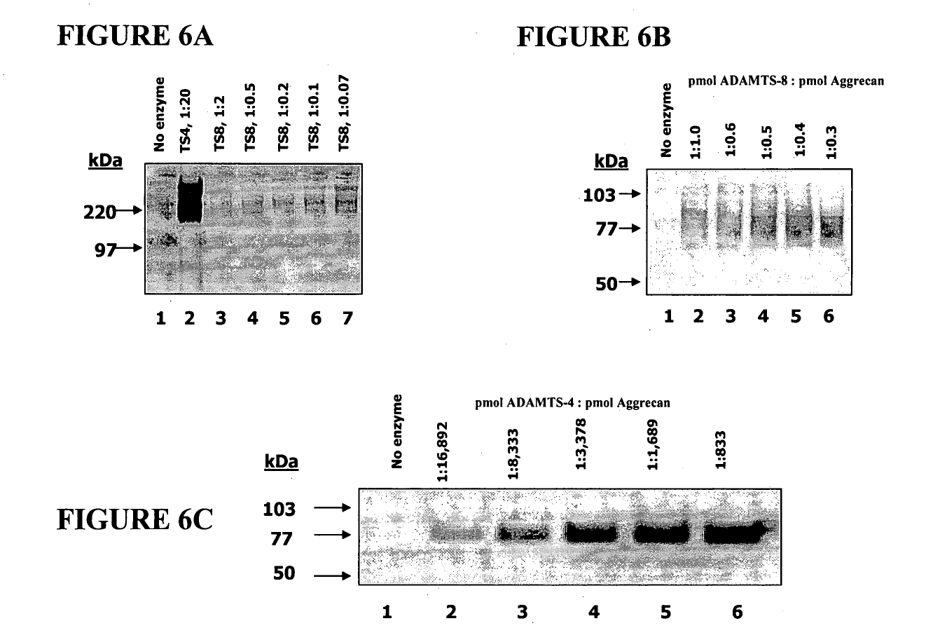

FIGS. 6A-6C demonstrate the results of the Western blot analyses of ADAMTS-4 and ADAMTS-8 digested aggrecan using MAb BC-3 and MAb AGG-C1. FIG. 6A shows the Western blot using MAb BC-3. In lane 1, no enzyme was added. Lane 2 shows ADAMTS-4 digested aggrecan at an enzyme:substrate molar ratio of 1:20. Lanes 3-7 show ADAMTS-8 digested aggrecan at an enzyme:substrate molar ratio of 1:2, 1:0.5, 1:0.2, 1:0.1, and 1:0.07, respectively. MAb BC-3 immunoreactive bands increased in intensity with increasing amounts of ADAMTS-8 protein relative to aggrecan substrate (FIG. 6A, lanes 3-7), indicative of aggrecan cleavage at the OA relevant position. However, a greater amount of enzyme relative to substrate was required than when using ADAMTS4 (comparing lanes 3-7 to lane 2 in FIG. 6A).

FIG. 6B is the Western blot using AGG-C1. The relative molar ratio of enzyme:substrate in each digest is indicated. MAb AGG-C1 immunoreactive bands were shown in FIG. 6B using enzyme:substrate ratios ranging from 1:1 to 1:0.3. In the same assay, ADAMTS4 also produced MAb AGG-C1 immunoreactive bands, but at much lower enzyme:substrate ratios (FIG. 6C, lanes 2-6). The migration positions of globular protein standards are shown to the left of each blot.

As a negative control, Western blots of aggrecan (25 μg) digested with up to 2.5 μg of rhMMP-13 produced no immunoreactive peptides, demonstrating that MAb AGG-C1 does not recognize the neoepitope sequence .DIPEN³⁴¹ (SEQ ID NO:26) which is generated by MMP cleavage of aggrecan. Furthermore, aggrecan digested with MMP-13 at similar enzyme:substrate ratios used for ADAMTS-8 was immunoreactive with MAb BC-14, which recognizes the MMP-generated neoepitope sequence ³⁴²FFG. (SEQ ID NO:27) but was not recognized by MAb BC-3, which recognizes the aggrecanase-generated neoepitope sequence ³⁷³ARGXX. (SEQ ID NO:24).

Detailed procedures for Western blot analyses are set forth below. Bovine articular cartilage aggrecan was incubated with purified ADAMTS-8 or ADAMTS-4 for 16 h at 37° C. in 50 mM Tris, pH 7.3, containing 100 mM NaCl and 5 mM CaCl₂. Digestion products were deglycosylated by incubation for 2 h at 37° C. in the presence of chondroitinase ABC (Seikagaku America, Falmouth, Mass.; 1 mU/μg aggrecan), keratanase (Seikagaku; 1 mU/μg aggrecan) and keratanase II (Seikagaku; 0.02 mU/μg aggrecan). Digestion products were separated on 4-12% Bis-Tris NuPAGE SDS PAGE gels (Invitrogen, Carlsbad, Calif.) and then electrophoretically transferred to nitrocellulose. Immunoreactive products were detected by Western blotting with MAb AGG-C1 (0.04 μg/ml) or MAb BC-3 (Caterson, et al., supra). Alkaline-phosphatase-conjugated secondary goat anti-mouse IgG (Promega Corp., Madison, Wis.; 1:7500) was subsequently incubated with the membranes, and NBT/BCIP substrate (Promega) was used to visualize immunoreactive bands. All antibody incubations were performed for 1 h at room temperature, and the immunoblots were incubated with the substrate for 5-15 min at room temperature to achieve optimum color development.

Other than ADAMTS4 (Aggrecanase 1) and ADAMTS5 (Aggrecanase 2), two other ADAMTS family members (ADAMTS1 and ADAMTS9) are reportedly capable of cleaving cartilage aggrecan somewhere within the protein, and both of them group in the same node on the phylogenetic tree as Aggrecanase 1, Aggrecanase 2, and ADAMTS-8. FIGS. 6A-6C show that the efficiency of ADAMTS-8's activity as an aggrecanase is comparable to that of these other ADAMTS family members. In addition, ADAMTS-8 aggrecanase activity appears to be specific for the Glu³⁷³-Ala³⁷⁴ site, because BC-3 Western blots (monitoring generation of the C-terminal aggrecan cleavage fragment) and AGG-C1 Western blots (monitoring generation of the N-terminal cleavage fragment) of aggrecan digested with recombinant human ADAMTS-8 show that the appropriate neoepitope is created by ADAMTS-8 treatment, and both aggrecan fragments that are generated appear to remain intact and are not further degraded, indicating a specific cleavage within the G1-G2 interglobular domain of aggrecan.

FIGS. 6A-6C also demonstrate that cleavage of bovine articular cartilage aggrecan by ADAMTS-8 at an enzyme:substrate ratio of 1:0.5 using the BC-3 neoepitope MAb and perhaps even lower using the AGG-C1 neoepitope MAb can be readily detected. This efficiency of cleavage at the aggrecan Glu³⁷³-Ala³⁷⁴ peptide bond compares favorably with aggrecanase activities reported for ADAMTS-1 and ADAMTS-9.

The comparison of ADAMTS-8 to ADAMTS-4 cleavage of aggrecan on the same Western blots revealed that ADAMTS-8 appeared to be less efficient than ADAMTS4 in cleaving cartilage aggrecan at the Glu³⁷³-Ala³⁷⁴ peptide bond under the test conditions. It has been suggested that carboxy-terminal proteolytic processing of ADAMTS4 may play a role in activating its proteolytic activity and mobilizing the enzyme by removing the putative C-terminal ECM-binding domains from the catalytic domain and reducing its affinity for GAG's present in the extracellular matrix. Thus, the possibility exists that ADAMTS-8 enzymatic activity may be inhibited by the persistent presence of the C-terminal domains, and that C-terminally truncated ADAMTS-8 may show enhanced aggrecanase activity. To address this question, a modified ADAMTS-8 cDNA, in which the coding sequence for the C-terminal thrombospondin and spacer domains was deleted, was constructed and expressed. This recombinant C-terminally truncated ADAMTS-8 was efficiently expressed and secreted, and the purified protein was active as judged by α2-macroglobulin assay, but it seemed to be no more active than full-length recombinant ADAMTS-8 on aggrecan substrate as judged by AGG-C1 Western blotting. However, the ability of ADAMTS-8 to retain its C-terminal GAG-binding domains may render ADAMTS-8 more efficient at cleaving cartilage aggrecan in vivo by keeping the enzyme localized to the cartilage matrix and thereby increasing the effective concentration of the enzyme. The presence of ADAMTS-8 mRNA in both normal and osteoarthritic human articular cartilage (FIG. 4) lends further support to the possibility that ADAMTS-8 functions as an aggrecanase in vivo.

Other related hyaluronan-binding proteoglycans such as neurocan, brevican, or versican may be cleaved more efficiently by ADAMTS-8. ADAMTS-8 mRNA is readily detectable in various subsections of brain, coincident with the expression patterns for neurocan and brevican. Murine ADAMTS-8 was first described as Meth2, one of two ADAMTS family members (ADAMTS-1 was the other) that was shown to be inhibitory in angiogenesis assays (Vázquez, et al., supra). One of the few and most abundant sites of ADAMTS-8 mRNA expression is aorta, a tissue rich in versican. Versican is a important vascular extracellular matrix protein with diverse roles in cellular adhesion, proliferation, and migration. Thus, it is tempting to speculate that ADAMTS-8 might function as a versicanase in the endothelium, possibly cleaving versican after the G1 domain and releasing it from the matrix. Such ADAMTS-8-mediated loss of versican from proliferating endothelial cells may explain the observed anti-angiogenic activity of ADAMTS-8. Supporting this possibility is the observation that fragments of aortic versican that are cleaved at the Glu⁴⁴¹-Ala⁴⁴² bond are found in vivo, mirroring the cleavage specificity for ADAMTS-8 that we show in this study. Versicanase activity has already been shown for ADAMTS-1 and ADAMTS-4, increasing the likelihood that ADAMTS-8 may be capable of cleaving versican with some level of efficiency and specificity.

### Example 11

### Expression Vectors

The mammalian expression vector pMT2 CXM, which is a derivative of p91023(b), can be used in the present invention. The pMT2 CXM vector differs from p91023(b) in that the former contains the ampicillin resistance gene in place of the tetracycline resistance gene and further contains an Xho I site for insertion of cDNA clones. The functional elements of pMT2 CXM include the adenovirus VA genes, the SV40 origin of replication (including the 72 bp enhancer), the adenovirus major late promoter (including a 5′ splice site and the majority of the adenovirus tripartite leader sequence present on adenovirus late mRNAs), a 3′ splice acceptor site, a DHFR insert, the SV40 early polyadenylation site (SV40), and pBR322 sequences needed for propagation in E. coli.

Plasmid pMT2 CXM is obtained by EcoR I digestion of pMT2-VWF, which has been deposited with the American Type Culture Collection (ATCC), Rockville, Md. (USA) under accession number ATCC 67122. EcoR I digestion excises the cDNA insert present in pMT2-VWF, yielding pMT2 in linear form which can be ligated and used to transform E. coli HB 101 or DH-5 to ampicillin resistance. Plasmid pMT2 DNA can be prepared by conventional methods. pMT2 CXM is then constructed using loopout/in mutagenesis. This removes bases 1075 to 1145 relative to the Hind III site near the SV40 origin of replication and enhancer sequences of pMT2. In addition, it inserts a sequence containing the recognition site for the restriction endonuclease Xho I. A derivative of pMT2CXM, termed pMT23, contains recognition sites for the restriction endonucleases Pst I, EcoR I, Sal I and Xho I. Plasmid pMT2 CXM and pMT23 DNA may be prepared by conventional methods.

pEMC2β1 derived from pMT21 may also be suitable in practice of the present invention. pMT21 is derived from pMT2 which is derived from pMT2-VWF. As described above, EcoR I digestion excises the cDNA insert present in pMT-VWF, yielding pMT2 in linear form which can be ligated and used to transform E. Coli HR 101 or DH-5 to ampicillin resistance. Plasmid pMT2 DNA can be prepared by conventional methods.
pMT21 is derived from pMT2 through the following two modifications. First, 76 bp of the 5′ untranslated region of the DHFR cDNA including a stretch of 19 G residues from G/C tailing for cDNA cloning is deleted. In this process, Pst I, EcoR I, and Xho I sites are inserted immediately upstream of DHFR.

Second, a unique Cla I site is introduced by digestion with EcoR V and Xba I, treatment with Klenow fragment of DNA polymerase I, and ligation to a Cla I linker (CATCGATG). This deletes a 250 bp segment from the adenovirus associated RNA (VAI) region but does not interfere with VAI RNA gene expression or function. pMT21 is digested with EcoR I and Xho I, and used to derive the vector pEMC2B1.

A portion of the EMCV leader is obtained from pMT2-ECAT1 by digestion with EcoR I and Pst I, resulting in a 2752 bp fragment. This fragment is digested with Taq I yielding an EcoR I-Taq I fragment of 508 bp which is purified by electrophoresis on low melting agarose gel. A 68 bp adapter and its complementary strand are synthesized with a 5′ Taq I protruding end and a 3′ Xho I protruding end.

The adapter sequence matches the EMC virus leader sequence from nucleotide 763 to 827. It also changes the ATG at position 10 within the EMC virus leader to an ATT and is followed by an Xho I site. A three way ligation of the pMT21 EcoR I-Xho I fragment, the EMC virus EcoR I-Taq I fragment, and the 68 bp oligonucleotide adapter Taq I-Xho I adapter resulting in the vector pEMC2β1.

This vector contains the SV40 origin of replication and enhancer, the adenovirus major late promoter, a cDNA copy of the majority of the adenovirus tripartite leader sequence, a small hybrid intervening sequence, an SV40 polyadenylation signal and the adenovirus VA I gene, DHFR and β-lactamase markers and an EMC sequence, in appropriate relationships to direct the high level expression of the desired cDNA in mammalian cells.

The construction of vectors may involve modification of the aggrecanase-related DNA sequences. For instance, a cDNA encoding an aggrecanase can be modified by removing the non-coding nucleotides on the 5′ and 3′ ends of the coding region. The deleted non-coding nucleotides may or may not be replaced by other sequences known to be beneficial for expression. These vectors are transformed into appropriate host cells for expression of the aggrecanase of the present invention.

In one specific example, the mammalian regulatory sequences flanking the coding sequence of aggrecanase are eliminated or replaced with bacterial sequences to create bacterial vectors for intracellular or extracellular expression of the aggrecanase molecule. The coding sequences can be further manipulated (e.g. ligated to other known linkers or modified by deleting non-coding sequences therefrom or altering nucleotides therein by other known techniques). An aggrecanase encoding sequence can then be inserted into a known bacterial vector using procedures as appreciated by those skilled in the art. The bacterial vector can be transformed into bacterial host cells to express the aggrecanases of the present invention. For a strategy for producing extracellular expression of aggrecanase proteins in bacterial cells, see, e.g. European Patent Application 177,343.

Similar manipulations can be performed for construction of an insect vector for expression in insect cells (see, e.g., procedures described in published European Patent Application 155,476). A yeast vector can also be constructed employing yeast regulatory sequences for intracellular or extracellular expression of the proteins of the present invention in yeast cells (see, e.g., procedures described in published PCT application WO86/00639 and European Patent Application 123,289).

A method for producing high levels of aggrecanase proteins in mammalian, bacterial, yeast, or insect host cell systems can involve the construction of cells containing multiple copies of the heterologous aggrecanase gene. The heterologous gene can be linked to an amplifiable marker, e.g., the dihydrofolate reductase (DHFR) gene for which cells containing increased gene copies can be selected for propagation in increasing concentrations of methotrexate (MTX). This approach can be employed with a number of different cell types.

For example, a plasmid containing a DNA sequence for an aggrecanase in operative association with other plasmid sequences enabling expression thereof and an DHFR expression plasmid (such as, pAdA26SV(A)3) can be co-introduced into DHFR-deficient CHO cells (DUKX-BII) by various methods including calcium phosphate-mediated transfection, electroporation, or protoplast fusion. DHFR expressing transformants are selected for growth in alpha media with dialyzed fetal calf serum, and subsequently selected for amplification by growth in increasing concentrations of MTX (e.g. sequential steps in 0.02, 0.2,1.0 and 5 μM MTX). Transformants are cloned, and biologically active aggrecanase expression is monitored by at least one of the assays described above. Aggrecanase protein expression should increase with increasing levels of MTX resistance. Aggrecanase polypeptides are characterized using standard techniques known in the art such as pulse labeling with ³⁵S methionine or cysteine and polyacrylamide gel electrophoresis. Similar procedures can be followed to produce other aggrecanases.

### Example 12

### Transfection of Expression Vectors

As one example an aggrecanase nucleotide sequence of the present invention is cloned into the expression vector pED6. COS and CHO DUKX B11 cells are transiently transfected with the aggrecanase sequence by lipofection (LF2000, Invitrogen) (+/−co-transfection of PACE on a separate PED6 plasmid). Duplicate transfections are performed for each molecule of interest: (a) one transfection set for harvesting conditioned media for activity assay and (b) the other transfection set for 35-S-methionine/cysteine metabolic labeling.

On day one, media is changed to DME(COS) or alpha (CHO) media plus 1% heat-inactivated fetal calf serum +/−100 μg/ml heparin on wells of set (a) to be harvested for activity assay. After 48 h, conditioned media is harvested for activity assay.

On day 3, the duplicate wells of set (b) are changed to MEM (methionine-free/cysteine free) media plus 1% heat-inactivated fetal calf serum, 100 μg/ml heparin and 100 μCi/ml 35S-methionine/cysteine (Redivue Pro mix, Amersham). Following 6 h incubation at 37° C., conditioned media is harvested and run on SDS-PAGE gels under reducing conditions. Proteins can be visualized by autoradiography.

The foregoing description of the present invention provides illustration and description, but is not intended to be exhaustive or to limit the invention to the precise one disclosed. Modifications and variations are possible consistent with the above teachings or may be acquired from practice of the invention. Thus, it is noted that the scope of the invention is defined by the claims and their equivalents.

1. A method for cleaving a proteoglycan, comprising contacting said proteoglycan with an isolated ADAMTS-8 protein which cleaves said proteoglycan.

2. The method according to claim 1, wherein said proteoglycan is an aggrecan molecule.

3. The method according to claim 2, wherein said ADAMTS-8 protein is a mature ADAMTS-8 protein.

4. The method according to claim 3, wherein said mature ADAMTS-8 protein is encoded by GenBank Accession No. AF060153 but lacks signal peptide and prodomain.

5. The method according to claim 3, wherein said mature ADAMTS-8 protein comprises amino acids 214-890 of SEQ ID NO:28.

6. A method for cleaving a proteoglycan, comprising contacting said proteoglycan with an isolated protease to cleave said proteoglycan, wherein said protease comprises an ADAMTS-8 metalloprotease catalytic domain.

7. The method of claim 6, wherein said proteoglycan is an aggrecan molecule.

8. The method of claim 7, wherein said ADAMTS-8 metalloprotease catalytic domain consists of amino acids 214-439 of SEQ ID NO:28.

9. The method of claim 7, wherein said protease comprises amino acids 214-588 of SEQ ID NO:28.

10. A method for cleaving a proteoglycan, comprising expressing a protease from a recombinant expression vector, wherein said protease comprises an ADAMTS-8 metalloprotease catalytic domain, and said protease cleaves said proteoglycan.

11. The method of claim 10, wherein said proteoglycan is an aggrecan molecule, and said recombinant expression vector is expressed in a mammalian cell which secretes said protease.

12. The method of claim 11, wherein said recombinant expression vector comprises a sequence encoding amino acids 214-890 of SEQ ID NO:28.

13. The method of claim 11, wherein said recombinant expression vector comprises a sequence encoding amino acids 214-588 of SEQ ID NO:28.

14. A method for identifying an agent capable of modulating an aggrecan cleavage activity of an ADAMTS-8 protein, said method comprising:
contacting said ADAMTS-8 protein with an aggrecan molecule in the presence or absence of said agent; and
measuring the aggrecan cleavage activity of said ADAMTS-8 protein in the presence or absence of said agent,
wherein a change in the aggrecan cleavage activity in the presence of said agent, as compared to in the absence of said agent, indicates that said agent is capable of modulating said aggrecan cleavage activity.

15. A pharmaceutical composition comprising said agent identified according to the method of claim 14.

16. A method for treating an aggrecan cleavage abnormality in a mammal, comprising administering said agent identified according to the method of claim 14 to said mammal.

17. A method for identifying an agent capable of modulating an aggrecan cleavage activity of an ADAMTS-8 protein, said method comprising:
contacting a protease with an aggrecan molecule in the presence or absence of said agent, said protease comprising an ADAMTS-8 metalloprotease catalytic domain and possessing the aggrecan cleavage activity; and
measuring the aggrecan cleavage activity of said protease in the presence or absence of said agent,
wherein a change in the aggrecan cleavage activity in the presence of said agent, as compared to in the absence of said agent, indicates that said agent is capable of modulating said aggrecan cleavage activity.

18. A method for modulating an aggrecan cleavage activity in an extracellular region of a mammalian cell, comprising inhibiting the expression of ADAMTS-8 in said mammalian cell.

19. The method of claim 18, wherein said inhibiting comprises introducing into said mammalian cell a polynucleotide which comprises or encodes an ADAMTS-8 RNAi or antisense sequence.

20. A method for treating an aggrecan cleavage abnormality in a mammal, comprising inhibiting the expression of ADAMTS-8 in selected cells of said mammal.


SEQUENCE LISTING (28 sequences)

SEQ ID NO: 1 - protein, 31 aa, Homo sapiens
GYIEAAVIPAGARRIRVVEDKPAHSFLALKD

SEQ ID NO: 2 - DNA, 29 bp, Homo sapiens
atgttccccgcccccgccgccccccggtg

SEQ ID NO: 3 - DNA, 29 bp, Homo sapiens
ggatcccccgaggcgctcgatcttgaact

SEQ ID NO: 4 - DNA, 24 bp, Homo sapiens
ggatccggccgggcgaccgggggc

SEQ ID NO: 5 - DNA, 30 bp, Homo sapiens
ctctagaagctctgtgagatacatggcgct

SEQ ID NO: 6 - DNA, 65 bp, Homo sapiens
ctctagacggcgggcacggagactgtctcctggatgcccctggtgcggccctgcccctccccaca

SEQ ID NO: 7 - DNA, 67 bp, Homo sapiens
acgtgtatttgacttttggggggaagacctcgccagggactgtcaggagctgcactgtcagaggctc

SEQ ID NO: 8 - DNA, 35 bp, Homo sapiens
cacacgttctttgttcctaatgacgtggactttag

SEQ ID NO: 9 - DNA, 32 bp, Homo sapiens
gcggccgctcacagggggcacagctggctttc

SEQ ID NO: 10 - protein, 5 aa, Homo sapiens
GSGSA

SEQ ID NO: 11 - protein, 8 aa, Homo sapiens
WSHPQFEK

SEQ ID NO: 12 - DNA, 24 bp, Homo sapiens
cttctagacggcgggcacggagac

SEQ ID NO: 13 - DNA, 82 bp, Homo sapiens
ttctagagcggccgccttatttttcgaactgcgggtggctccaagcagatccggatcccagggggcatagctggctttcgca

SEQ ID NO: 14 - DNA, 20 bp, Homo sapiens
ggaccgctgcaagttgttct

SEQ ID NO: 15 - DNA, 20 bp, Homo sapiens
ggacacagatggccagtgtt

SEQ ID NO: 16 - DNA, 25 bp, Homo sapiens
ccatcaatcaccttggcctcgaaca

SEQ ID NO: 17 - DNA, 20 bp, Homo sapiens
ccacatcgctcagacaccat

SEQ ID NO: 18 - DNA, 18 bp, Homo sapiens
gcgcccaatacgaccaaa

SEQ ID NO: 19 - DNA, 25 bp, Homo sapiens
gggaaggtgaaggtcggagtcaacg

SEQ ID NO: 20 - DNA, 63 bp, Homo sapiens
ggccagtgaattgtaatacgactcactatagggaggcggtttttttttttttttttttttttt

SEQ ID NO: 21 - protein, 13 aa, Homo sapiens
CGGPLPRNITEGE

SEQ ID NO: 22 - protein, 13 aa, Homo sapiens
GGLPLPRNITEGE

SEQ ID NO: 23 - protein, 23 aa, Homo sapiens
GGLPLPRNITEGEARGSVILTVK

SEQ ID NO: 24 - protein, 5 aa, Homo sapiens
  misc_feature (4)..(5): Xaa can be any naturally occurring amino acid
ARGXX

SEQ ID NO: 25 - protein, 6 aa, Homo sapiens
NITEGE

SEQ ID NO: 26 - protein, 5 aa, Homo sapiens
DIPEN

SEQ ID NO: 27 - protein, 3 aa, Homo sapiens
FFG

SEQ ID NO: 28 - protein, 890 aa, Homo sapiens
MLPAPAAPRWPPLLLLLLLLLLPLARGAPARPAAGGQASELVVPTRLPGSAGELALHLSAFGKGFVLRLAPDDSFLAPEFKIERLGGSGRATGGERGLRGCFFSGTVNGEPESLAAVSLCRGLSGSFLLDGEEFTIQPQGAGGSLAQPHRLQRWGPAGARPLPRGPEWEVETGEGQRQERGDHQEDSEEESQEEEAEGASEPPPPLGATSRTKRFVSEARFVETLLVADASMAAFYGADLQNHILTLMSVAARIYKHPSIKNSINLMVVKVLIVEDEKWGPEVSDNGGLTLRNFCNWQRRFNQPSDRHPEHYDTAILLTRQNFCGQEGLCDTLGVADIGTICDPNKSCSVIEDEGLQAAHTLAHELGHVLSMPHDDSKPCTRLFGPMGKHHVMAPLFVHLNQTLPWSPCSAMYLTELLDGGHGDCLLDAPRAALPLPTGLPGRMALYQLDQQCRQIFGPDFRHCPNTSAQDVCAQLWCHTDGAEPLCHTKNGSLPWADGTPCGPGHLCSEGSCLPEEEVERPKPVADGGWAPWGPWGECSRTCGGGVQFSHRECKDPEPQNGGRYCLGRRAKYQSCHTEECPPDGKSFREQQCEKYNAYNYTDMDGNLLQWVPKYAGVSPRDRCKLFCRARGRSEFKVFEAKVIDGTLCGPETLAICVRGQCVKAGCDHVVDSPRKLDKCGVCGGKGNSCRKVSGSLTPTNYGYNDIVTIPAGATNIDVKQRSHPGVQNDGNYLALKTADGQYLLNGNLAISAIEQDILVKGTILKYSGSIATLERLQSFRPLPEPLTVQLLTVPGEVFPPKVKYTFFVPNDVDFSMQSSKERATTNIIQPLLHAQWVLGDWSECSSTCGAGWQRRTVECRDPSGQASATCNKALKPEDAKPCESQLCPL